(19) United States
(12) Patent Application Publication
(10) Pub. No.: US 2026/0335895 A1
(43) Pub. Date: Oct. 29, 2026

(54) DIGITAL FERROFLUIDIC DEVICE AND METHOD FOR MULTIPLEXED ASSAYS AND VIRAL TESTING

(71) Applicant: THE REGENTS OF THE UNIVERSITY OF CALIFORNIA, Oakland, CA (US)

(72) Inventors: Sam Emaminejad, Los Angeles, CA (US); Dino Di Carlo, Los Angeles, CA (US); Haisong Lin, Los Angeles, CA (US); Wenzhuo Yu, Los Angeles, CA (US); Kiarash AmirMozafariSabet, Los Angeles, CA (US)

(73) Assignee: THE REGENTS OF THE UNIVERSITY OF CALIFORNIA, Oakland, CA (US)

(21) Appl. No.: 19/107,020

(22) PCT Filed: Aug. 31, 2023
(86) PCT No.: PCT/US2023/073249

§ 371 Date: Feb. 26, 2025

Related U.S. Application Data
(60) Provisional application No. 63/403,656, filed on Sep. 2, 2022.

(51) Int. Cl. B01L 3/00; C12Q 1/6844

(52) U.S. Cl. CPC B01L 3/502784; C12Q 1/6844; B01L 2200/14; B01L 2200/16; B01L 2300/027; B01L 2300/0627; B01L 2300/0816; B01L 2300/0861; B01L 2300/18; B01L 2400/043; C12Q 2600/16

(57) ABSTRACT

A ferrofluidic fluid assay device uses swarm of millimeter-sized magnets was employed as mobile robotic agents (“ferrobots”) for precise and robust handling of magnetized sample droplets and high-fidelity delivery of flexible assay workflows. Within a palm-sized printed circuit board-based programmable platform a myriad of sample handling and bioanalytical operations involved in pooled testing can be performed. This automated technology was applied using the loop-mediated isothermal amplification and detection of SARS-COV-2 virus in clinical samples, where the test results completely matched those obtained off-chip. This technology is easily manufacturable and distributable, and its adoption for viral testing could lead to a 10 to 300-fold reduction in reagent costs, and three orders of magnitude reduction in instrumentation cost. Multiplexed assays may also be performed on a single device.

### RELATED APPLICATION

This application claims priority to U.S. Provisional Patent Application No. 63/403,656 filed on Sep. 2, 2022, which is hereby incorporated by reference. Priority is claimed pursuant to 35 U.S.C. § 119 and any other applicable statute.

### TECHNICAL FIELD

The technical field generally relates to digital fluidic platforms. More specifically, the technical field relates to a digital fluidic platform that uses the electronic actuation of individual coils formed on or in a substrate to impart magnetic fields on moveable permanent magnets that are used to actuate magnetic droplets. The digital fluidic platform may be used to implement multiplexed and automated assays. The platform may also be used for pooled clinical sample testing of, for example, viruses.

### BACKGROUND

Over the past two decades, major epidemics (SARS, Zika, MERS, and Ebola) and pandemics (H1N1 and COVID-19) have emerged with increasingly alarming regularity. Although currently the world is grappling with the COVID-19 pandemic, the occurrence of the next wave of infectious disease outbreaks in the coming years is deemed inevitable, given the rise in population, urbanization, and global travel/trade. In that regard, large-scale population screening is the primary safeguard to contain epidemics, prevent pandemics, and mitigate their human and economic costs upon their onset.

Accordingly, increasing the viral diagnostic and surveillance testing capacity globally is central to epidemic/pandemic preparedness. Among the test options, nucleic acid amplification tests (NAATs) are advantageous over the antigen- and antibody-based counterparts, owing to their superior sensitivity, specificity, and ability for rapid deployment without the need to generate specific diagnostic antibodies. To perform NAATs at large scale and frequency, accessible automated testing platforms are required to analyze samples with high throughput, fast turnaround time, and minimal capital cost/reagent use. In particular, the strategic pooling of samples, when most patients are expected to be negative, can lead to a dramatic reduction in resource utilization amid pandemic-induced supply chain disruptions (outweighing the marginal risk of dilution-induced false negatives). Accordingly, flexible testing workflows dictated by adaptive pooling algorithms—such as viral prevalence-based ones—that are intended to maximize the screening efficiency are needed.

However, current automated NAAT-based testing platforms are unable to perform the integrated liquid handling, analysis, and automated feedback processes necessary to achieve these flexible workflows. Additionally, they employ bulky, expensive, and reagent-wasteful robotic liquid handlers and bio-instruments, with heavy installations and maintenance needs, and thus, they are restricted to centralized laboratory settings.

### SUMMARY

To enable adaptive pooled testing, an automated NAAT-based testing platform was created, which performs programmable liquid handling and bioanalytical operations within flexible workflows and in a parallel manner. Instead of resource-intensive, and functionally-limited robotic liquid handlers, a swarm of individually-addressable millimeter-sized magnets were employed as mobile robotic agents (“ferrobots”) that can manipulate magnetic nanoparticle-spiked droplets (“ferro-droplets”) with high precision and robustness. The seamless integration of fluidware, hardware, and software allowed for programing and streamlining the droplet-based operations, and delivering versatile automated NAAT-centered workflows within a compact platform (e.g., here a reverse transcription loop mediated isothermal amplification, RT-LAMP was implemented). To maximize the screening efficiency, a prevalence-based adaptive testing algorithm was formulated. This algorithm particularly determines the optimal testing mode and guides the operational workflow in accordance with a square matrix pooling scheme, without entailing overly burdensome sample handling procedures. Adopting this approach over the fixed individual testing approach (universally pursued) allows for significant savings over a wide viral prevalence range.

In one embodiment, a ferrofluidic fluid assay device includes a first substrate having a plurality of individually addressable coils formed therein or thereon; a second substrate disposed adjacent to the first substrate and separated by a gap, the second substrate containing therein one or more volumes of ferrofluid, one or more sample holding chambers or regions, one or more corrugated features for defining sub-volume(s) of the ferrofluid and/or sample, and one or more assay chambers holding an assay solution or assay reagent therein; one or more permanent magnets interposed in the gap formed between the first substrate and the second substrate, wherein the one or more permanent magnets are moveable within the gap; and a power source and control circuitry electrically connected to the individually addressable coils and configured to selectively drive current through one or more of the individually addressable coils to move the one or more permanent magnets and the one or more volumes of ferrofluid.

In another embodiment, a method of using the ferrofluidic fluid assay device includes the operations of: loading sample(s) into the one or more sample holding chambers or regions; and driving current through the one or more of the plurality of individually addressable coils to move the one or more permanent magnets to perform one or more unit operations on the one or more volumes of ferrofluid, wherein the unit operations comprise moving the one or more volumes of ferrofluid across a surface of the second substrate, merging the one or more volumes of ferrofluid with the sample(s), forming a plurality of sub-volumes of ferrofluid mixed with the sample with the one or more corrugated features, mixing the one or more volumes of ferrofluid and sample, pooling the one or more volumes of ferrofluid and sample, and moving the one or more volumes of ferrofluid and sample to the one or more assay chambers.

In another embodiment, the device is a handheld portable device that uses a microfluidic chip or cartridge that works with a first substrate or printed circuit board that is located within the device. The device uses one or more moveable permanent magnets to manipulate one or more volumes of ferrofluid. In this embodiment, the microfluidic chip or cartridge includes one or more trench chambers including a trench recess formed therein. The one or more trench chambers capture magnetic beads that are carried by the one or more volumes of ferrofluid that are used to capture target species located within a sample loaded in the device. The target species may include nucleic acids, proteins, other biomolecules, or the like. The magnetic beads enable the enrichment of nucleic acids or other target species from a larger sample into smaller reaction volumes that are then delivered to one or more assay chambers using moving permanent magnets. The one or more assay chambers are then optically interrogated to identify the presence or absence of the target species in the sample, or to quantify the abundance of the target species.

### BRIEF DESCRIPTION OF THE DRAWINGS

FIG. 1A illustrates a cross-sectional view of a ferrofluidic fluid handling device according to one embodiment.

FIG. 1B illustrates a view of the first substrate (i.e., printed circuit board (PCB)) according to one embodiment along with associated control electronics.

FIGS. 1C-1I illustrate an overview of the bioanalytical swarm ferrobotic device for accessible, adaptable, and automated viral testing. FIG. 1C illustrates spatio-temporal varying COVID-19 viral prevalence (based on the test positivity rate data from Our World in Data and California Health and Human Services Open Data Portal). FIG. 1D illustrates the required number of tests per person to find all infected people (across different levels of local viral prevalence), based on the square matrix pooled testing strategy. The highlighted curve (with arrow labeled as “adaptive testing”) illustrates that maximal screening efficiency can be achieved via adaptive (prevalence-based) testing. FIG. 1E is an illustration of the optimal testing modes and the associated ferrobotic microfluidic chips (scale bar: 1 cm) for the representative local viral prevalence levels of 25%, 10%, and 2%. FIG. 1F is a schematic overview of the automated workflows for individual and pooled testing of 16 samples. FIG. 1G is an exploded schematic of a representative ferrobotic viral testing platform. FIG. 1H are schematic illustrations of the ferrobotic equivalents of lab-based NAAT liquid handling operations, including aliquoting, merging and mixing. FIG. 1I is an optical image of a representative ferrobotic viral testing platform for 4² pooled testing.

FIGS. 2A-2J illustrate ferrobotic operations enable NAAT-based testing. FIG. 2A shows the characterization of maximum ferro-droplet transportation velocity within different oil environments. Inset shows overlaid sequential images, visualizing the transportation process (scale bar: 3 mm). Error bars, SE (n=4). FIG. 2B shows the characterization of the aliquoted droplet size for different corrugated opening widths (channel height: ~900 μm). Inset shows multiple aliquots of the same ferro-droplet source can be produced by extending the corrugated feature in an array format (scale bar: 5 mm). Error bars, SE (n=12 across 3 replicates). FIG. 2C shows the characterization of the threshold voltage for droplet merging using different concentrations of a surfactant (PicoSurf™) within an oil (Novec™) environment. Inset shows sequential optical images of the merging process (scale bar: 5 mm). Error bars, SE (n=3). FIG. 2D shows the characterization of the cyclic ferrobotic operations, involving aliquoting, merging, and intermediate transportation of a parent droplet to evaluate the robustness of the ferrobotic operations (performed for >800 cycles, scale bar: 3 mm). Parent droplet size varied by <1% for each of the post-merging and post-aliquoting states (characterized optically). FIG. 2E illustrates progressive mixing index for different actuation frequencies. Top: corresponding images of the merged droplets under mixing at different actuation frequencies for 15 s. FIG. 2F shows the characterization of the local temperature set by an on-board resistive heater for different input current. FIG. 2G is a schematic of the RT-LAMP reaction and detection mechanism. FIG. 2H is gel electrophoresis analysis of the RT-LAMP reaction product (reaction period: 30 min). FIGS. 2I and 2J are sequential optical images (2I) and on-chip readouts (2J) of the RT-LAMP assay performed in ferro-droplets containing negative control and spiked SARS-COV-2 positive control RNA (25, 100, 1000 cp/μL) samples. Error bars, SE (n=10).

FIGS. 3A-3E illustrate the performance of an automated ferrobotic SARS-COV-2 RT-LAMP workflow for individual clinical sample testing. FIG. 3A is an annotated image of the microfluidic chip for individual sample testing (scale bar: 5 mm). FIG. 3B illustrates the timeline of the streamlined on-chip operations for automated individual testing, which includes active ferrobotic sample processing operations over a time window of 1.75 min. Heat lysis and RT-LAMP reaction are correspondingly performed at 95 and 65 °C. FIG. 3C shows sequential optical images of the active ferrobotic sample processing operations (performed automatically). FIG. 3D is a comparison of the ferrobotic SARS-COV-2 RT-LAMP assay readouts with the corresponding RT-PCR results (Ct values) for a collection of one hundred clinical samples. Each datapoint represents one sample. Inset compares ferrobotically-produced vs. manually-performed RT-LAMP assay results, illustrating that the corresponding sample test results are in complete agreement (for the same collection of samples). FIG. 3E is the corresponding ROC curve of the analyzed samples. The sensitivity and specificity are based on the set cut-off value of 710 a.u. (also serving as the on-chip detection threshold).

FIGS. 4A-4F illustrate the workflow used to perform a pooled SARS-COV-2 RT-LAMP using a ferrobot swarm. FIG. 4A is a schematic of the square matrix pooling scheme. The flow chart at the center provides an overview of the infected sample identification process based on the assay pooled (A) or row/column (Rᵢ/Cⱼ) responses. FIG. 4B shows sequential optical images of an automated 4² pooling workflow performed by a team of nine ferrobots. To combine aliquots in each pooling step, they are ferrobotically collected, merged, mixed, and then dispensed as a 1-μL droplet. Inset images show the critical intermediary ferrobotic operations. FIGS. 4C, 4D are optical images and readouts obtained from ferrobotic pooled testing of two groups of 9 clinical samples using the 3² pooling chip. The negative assay A response indicated no infected sample was present among the first group of samples in (FIG. 4C). The positive assay A response along with the positive assay R₂ and C₂ responses led to the identification of the infected sample (located at the 2^{nd} row/2^{nd} column) among the second group of samples (FIG. 4D). FIGS. 4E, 4F show optical images and readouts obtained from ferrobotic pooled testing of two groups of 16 clinical samples using the 4² pooling chip. The negative assay A response indicated no infected sample was present among the first group of samples in (FIG. 4E). The positive assay A response along with the positive assay R₃ and C₃ responses led to the identification of the infected sample (located at the 3^{rd} row/3^{rd} column) among the second group of samples (FIG. 4F). Error bars indicate different trials of optical reading, SE (n=10).

FIGS. 5A-5D illustrate the fabrication of microfluidic chips and characterization of ferrobotic operations. FIG. 5A shows a fabrication process of the microfluidic chip (e.g., individual testing chip). FIG. 5B shows optical images of ferro-droplet-carrying ferrobots at Δt=0.25 s, visualizing the maximum achievable transportation velocities within different oil environments. In all cases, the ferrobots are programmed to travel at the highest possible velocity that allows for the ferro-droplets to keep up with the underlying ferrobot movement. FIG. 5C illustrates optical images of the arrays of aliquoted droplets for different corrugated opening widths. FIG. 5D illustrates the relationship between the ferro-droplet diameter and volume. Insets show optical images of the ferro-droplets.

FIGS. 6A-6B show the characterization of the robustness of ferrobotic actuation over a range of ferro-droplet ionic strengths and chemical conditions. FIG. 6A illustrates a schematic of the characterized ferrobotic actuation, involving cyclic actuation of 10 ferro-droplets (with differing ionic strengths and chemical conditions) by designated ferrobots (12 commuted pixels-per-cycle/ferrobot). The actuation events and commuted pixels are tracked by monitoring the current through impedance sensing electrode pairs. FIG. 6B illustrates corresponding normalized measured current through the electrode pairs and accumulated commuted pixels over time (based on the recorded current peaks) are shown, resulting in a total of >8 million actuation events over >24 h (only limited by observation time).

FIGS. 7A-7F show a demonstration of a fully integrated platform and component characterization. FIG. 7A is an optical image of a representative multi-sample ferrobotic platform for individual testing (here, 32 test sites). FIG. 7B is an exploded schematic of the microfluidic layer and actuation/sensing layer for a ferrobotic viral testing device designed for individual testing. FIG. 7C illustrates the programmed input current profiles of two neighboring (12-mm apart) on-board resistive heaters and the correspondingly established local temperature profiles. FIG. 7D shows the characterization of optical sensor readout versus standard Nanodrop readout (using solutions containing different concentrations of a pink color dye). FIG. 7E is a schematic diagram of the circuitry for a multi-sample individual testing PCB (extended into an array format). FIG. 7F is a schematic diagram of the control circuitry used for multiplexed testing (featuring an expanded coiled navigation floor).

FIGS. 8A-8E show a characterization of the colorimetric RT-LAMP assay. FIG. 8A illustrate a quantification of the RT-LAMP assay response by real-time absorbance measurement using a plate reader. FIG. 8B shows the absorbance spectra and corresponding optical images (insets) of the RT-LAMP reaction products for different volumes of positive input samples (6 to 1 μL, reaction period: 30 min). FIG. 8C shows the absorbance spectra of the RT-LAMP assay solution pre- and post-RT-LAMP reaction, showing the utility of the assay pH adjustment for optimizing the assay's colorimetric response. FIGS. 8D and 8E show RT-LAMP colorimetric readout for ferro-droplet sample volumes of 1 μL (FIG. 8D) and 100 nL (FIG. 8E), across various input SARS-COV-2 RNA copy numbers or negative controls (10 independent tests per condition). Limits of detection of 25 cp/μl and 50 cp/μl are observed for the 1 μL and 100 nL sample respectively, likely limited by statistical sampling errors when copy number approaches ~1 per sample volume. Error bars, SE (n=5).

FIGS. 9A-9B illustrate the control and execution of the ferrobotic operations at software & hardware levels. A software-programmable Arduino-based microcontroller (FIG. 9A), which hosts the installed ferrobotic instruction sets (FIG. 9A), is interfaced with a bank of switching ICs to selectively activate the addressable electromagnetic coils, and subsequently, direct the ferrobots to perform the required operations which are illustrated in FIG. 9B.

FIGS. 10A-10B illustrate the repeatability and reproducibility of ferrobotic tests with clinical samples. Ferrobotic SARS-COV-2 RT-LAMP assay readouts corresponding to ferro-droplet sample volumes of 1 μL (FIG. 10A) and 100 nL (FIG. 10B) are illustrated. Clinical samples from 10 SARS-COV-2 infected and 10 uninfected patients (pre-characterized via RT-PCR) were analyzed across three replicates for each sample volume. Error bars indicate different repeats of colorimetric reading, SE (n=5). The colorimetric detection threshold for positivity for the 1 μL- and 100 nL-test sample volume cases is correspondingly 710 a.u. and 890 a.u. in this system.

FIGS. 11A-11D illustrate how the device may be used in ferrobotically-automated RT-LAMP for multiplexed testing. FIG. 11A shows an annotated image of the microfluidic chip for multiplexed testing. FIG. 11B shows sequential optical images of the active ferrobotic sample processing operations (performed automatically). FIG. 11C shows the timeline of the streamlined on-chip operations for automated multiplexed testing, which includes active ferrobotic sample processing operations over a time window of 2:20 min:s.

FIG. 11D illustrates optical images and corresponding on-chip readouts of the SARS-COV-2, Influenza H1N1, and rActin (internal control, IC) RT-LAMP assays for different combinations of input samples (spiked with corresponding RNAs). Nasal Swab (NS) samples naturally contain rActin. Error bars indicate different trials of optical reading, SE (n=10).

FIG. 12 illustrates and example of sample analysis and interpretation flowchart for pooled testing.

FIGS. 13A-13F illustrate microfluidic chip layouts and the ferrobot navigation plans and task assignment for pooled testing. FIGS. 13A and 13B show schematic illustrations of the microfluidic chip layout for 3² (FIG. 13A) and 4² (FIG. 13B) pooled testing (key features are outlined and labeled). FIGS. 13C and 13D illustrate an overview of the navigation plans of seven and nine ferrobots for 3² (FIG. 13C) and 4² (FIG. 13D) pooled testing. Each Fᵢ represents an individual ferrobot. FIGS. 13E and 13F illustrate the timeline of the ferrobots' assigned tasks and status (active, standby) for 3² (FIG. 13E) and 4² (FIG. 13F) pooled testing.

FIGS. 14A-14C illustrate the detection of virus in diluted and undiluted clinical samples by colorimetric SARS-COV-2 RT-LAMP assay. FIGS. 14A, 14B show optical images of the standard assay responses in microfuge tubes (FIG. 14A) and on-chip assay responses (FIG. 14B) for undiluted or diluted patient samples with different Ct values. FIG. 14C shows corresponding optical readouts of the on-chip assay responses.

FIG. 15: Characterization of the aliquoted droplet volume for different corrugated opening widths (channel height: ~150 μm). Error bars, SE (n=12 across 4 replicates).

FIG. 16A illustrates optical absorbance readouts of the colorimetric RT-LAMP assay performed at 60 °C, 65 °C, and 70 °C using a SARS-COV-2 positive control (PC, 100 cp/μL) and negative control (NC) samples.

FIG. 16B shows the characterization of the local temperature set by an on-board resistive heater for different input current at different surrounding temperatures (~15 °C, 25 °C, 35 °C, generated by a Peltier module). Quadratic relationship indicates temperature increase by Joule heating.

FIG. 16C shows the required input current through the on-board resistive heater to maintain a local temperature of 65 °C for different surrounding temperatures (derived from the results shown in FIG. 16B).

FIG. 17 illustrates images and optical absorbance readouts of the RT-LAMP assay performed in ferro-droplets containing: negative control, negative control spiked with E. coli, SARS-COV-2 positive control RNA, and SARS-COV-2 positive control RNA spiked with E. coli (PC: 10,000 cp/μL; E. coli: 10,000 CFU/μL).

FIG. 18 shows sequential images of the active ferrobotic sample processing operations in a small sample volume device (dispensed sample~100 nL; scale bar: 5 mm).

FIGS. 19A-19D illustrate pooled testing using 100 nL sample volumes. FIGS. 19A, 19B show readouts obtained from ferrobotic pooled testing of two groups of 9 clinical samples using the 3² pooling chip. The negative assay A response indicated no infected sample was present among the first group of samples in (FIG. 19A). The positive assay A response along with the positive assay Rᵢ and C₂ responses led to the identification of the infected sample (located at the 1^{st} row/2^{nd} column) among the second group of samples (FIG. 19B). FIGS. 19C and 19D show readouts obtained from ferrobotic pooled testing of two groups of 16 clinical samples using the 4² pooling chip. The negative assay A response indicated no infected sample was present among the first group of samples in (FIG. 19C). The positive assay A response along with the positive assay R₄ and C₃ responses led to the identification of the infected sample (located at the 4th row/3rd column) among the second group of samples (FIG. 19D). Error bars indicate repeated optical readings, SE (n=10). Assays were performed in small volume microfluidic chips (channel height: ~150 μm; aliquoted sample volume: 100 nL; and reagent volume: 1.9 μL).

FIG. 20 illustrates the maximum ferro-droplet velocity within different oil environments is plotted as a function of oil viscosity.

FIG. 21A illustrates a schematic of the relevant pressure mechanisms acting at the interface of two oppositely charged emulsion droplets during coalescence.

FIG. 21B shows the estimated threshold disjoining pressure across different surfactant concentrations.

FIGS. 22A-22C illustrate a comparison of conventional PCB electromagnetic coil-based actuation vs. ferrobotic actuation. FIG. 22A shows a simulated magnetic flux density induced by a standalone electromagnetic coil. FIG. 22B shows simulated magnetic flux density induced ferrobotically. FIG. 22C illustrates magnetic flux density enhancement factor due to the presence of the field-amplifying ferrobot. The x axis is the vertical distance from the center of the magnetic source.

FIG. 23A illustrates the experimental setup for the standard ferrobotic system including a power supply, laptop, an Arduino microcontroller (MCU) module, and the ferrobotic control unit.

FIG. 23B illustrates a battery-operable handheld ferrobotic unit with 7.4 V- and 3.7 V-lithium-ion batteries and MCU module integrated on its backside. These components are placed next to a quarter-dollar coin for comparison. Overlaid sequential images derived from video frames that follow actuation of the ferro-droplets via moving ferrobots on the handheld ferrobotic system.

FIGS. 24-1, 24-2, 24-3, 24-4, 24-5, 24-6 include a table showing clinical sample results.

FIG. 25A illustrates another embodiment of the digital ferrofluidic fluid assay device. This is a self-contained, handheld device that is able to perform assays such as viral assays.

FIG. 25B illustrates an exemplary workflow for the trench chamber illustrated in FIG. 25A.

FIG. 25C illustrates a three-dimensional (3D) view of a single droplet of ferrofluid and reagents located above a ferrobot traveling over a PCB containing addressable coils.

FIG. 26A illustrates the trench recess in the trench chamber acts as a barrier to magnetic beads and traps the same inside the trench chamber as the ferrofluid volume or droplet is moved past the trench chamber.

FIG. 26B illustrates a graph showing separation efficiency for different magnetic bead concentrations. High separation efficiencies are achieved over a range of magnetic bead concentrations.

### DETAILED DESCRIPTION OF ILLUSTRATED EMBODIMENTS

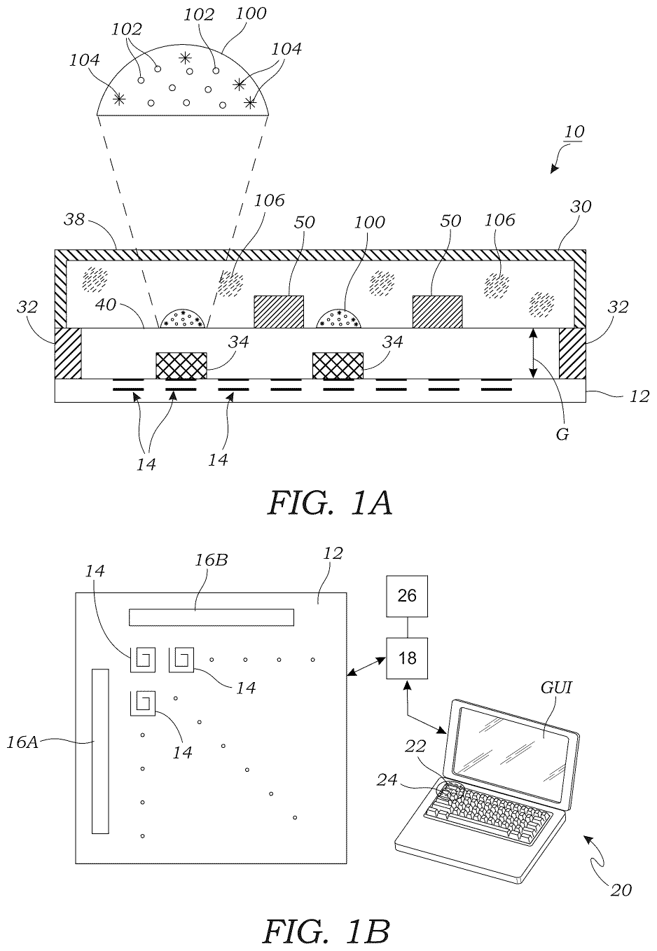

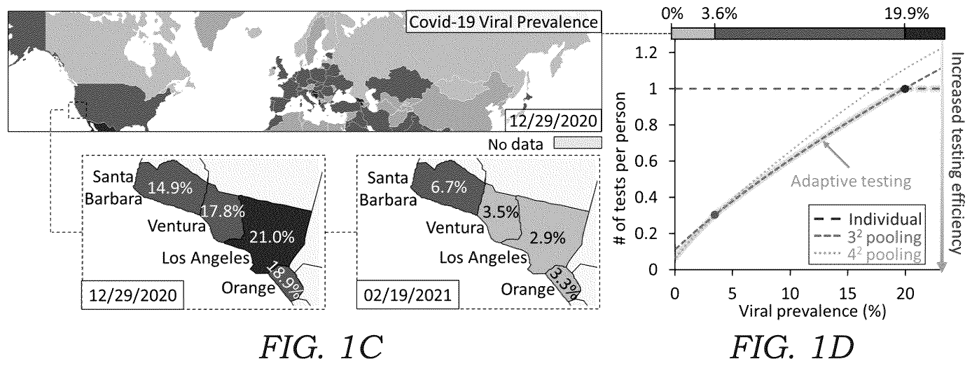

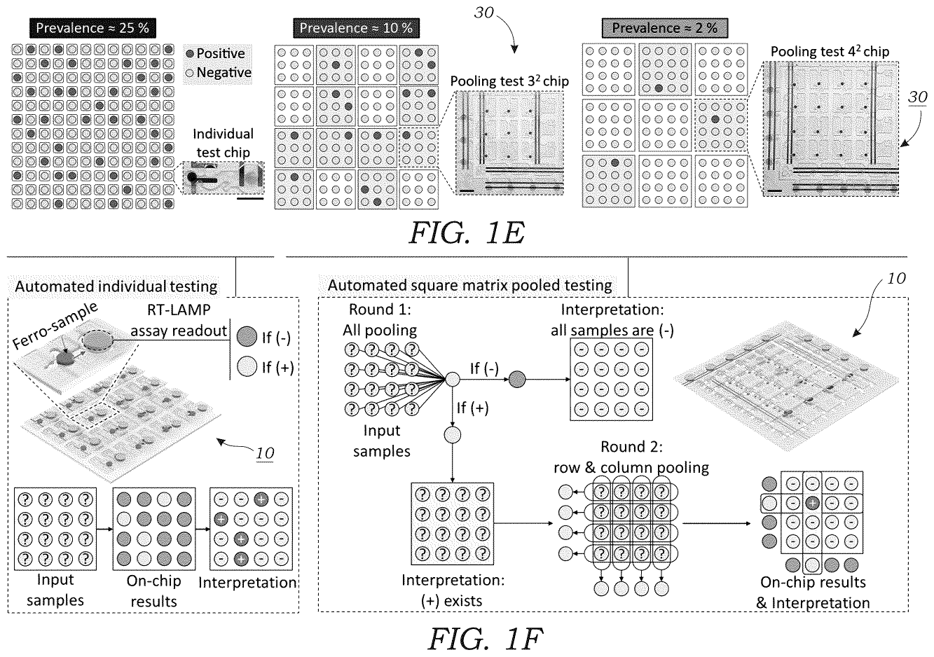

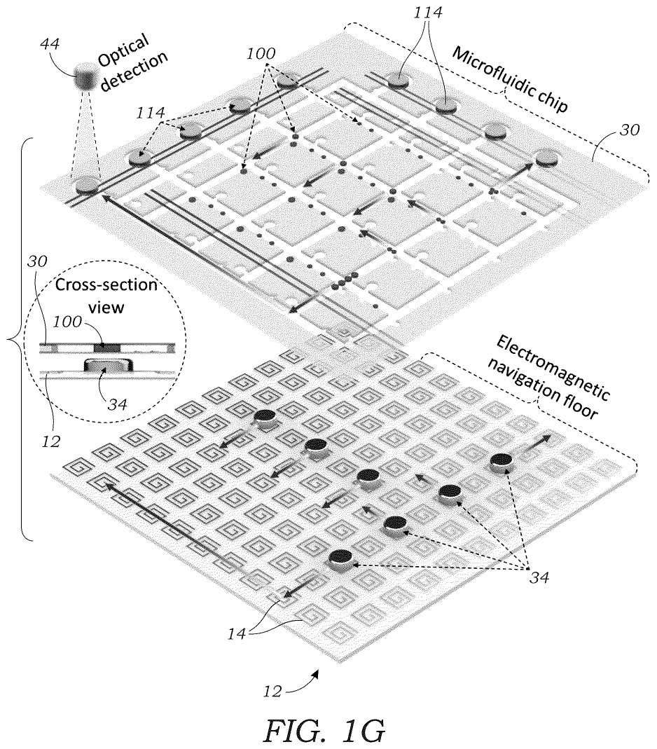

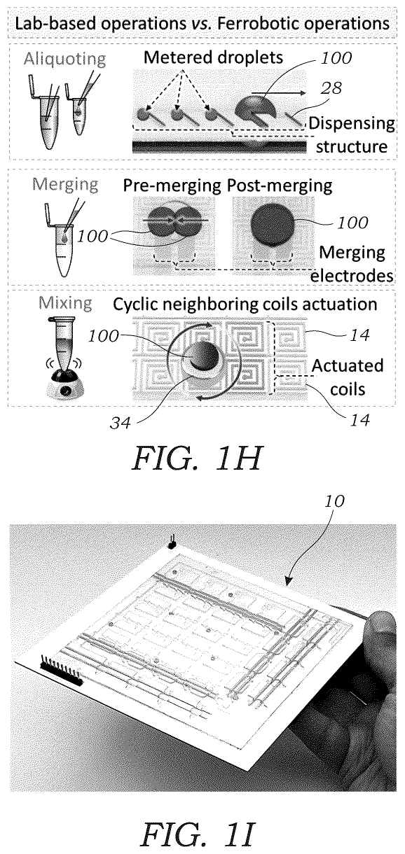

FIG. 1A illustrates a cross-sectional view of a digital ferrofluidic fluid assay device 10 according to one embodiment. The ferrofluidic fluid assay device 10 is “digital” in the sense that it creates, manipulates, and operates on discrete volumes or droplets of ferrofluid 100 contained within the ferrofluidic fluid assay device 10. A ferrofluid 100 is a liquid fluid that is magnetic due to the presence of small (e.g., nanometer-sized to tens of nanometer-sized) magnetic particles 102 suspended in a carrier fluid. The volumes or droplets of ferrofluid 100 may also contain a package 104 that may be sample, reagent, or the like. The package 104 may be a biological material resulting in a bio-package 104.

The ferrofluidic fluid assay device 10 includes a first substrate 12 that has a plurality of individually addressable coils 14 formed therein or thereon. The individually addressable coils 14 operate as an electromagnet (EM) when actuated and current is driven through the addressable coils 14. This first substrate 12 acts as a navigation floor for permanent magnets 34 which act or operate as “ferrobots” as explained herein. The first substrate 12 may, in one preferred embodiment, be a printed circuit board (PCB) that includes the plurality of individually addressable coils 14 formed therein. In one preferred embodiment, the first substrate 12 is formed from a multi-layer PCB where the plurality of individually addressable coils 14 are formed as spirals with different layers of the PCB 12 containing additional spirals of the coil structure (e.g., three different layers for the spiral structure). FIG. 1B illustrates a view of the navigation floor or first substrate 12 (e.g., PCB) according to one embodiment. The plurality of individually addressable coils 14 are formed as an array or matrix on the first substrate 12 with individual addressable coils 14 formed in rows and columns, although other configurations may be used. The number of individual addressable coils 14 may vary depending on the size of the overall ferrofluidic fluid assay device 10, size of individual addressable coils 14, pitch between adjacent coils 14, etc. In some embodiments, greater than 100 addressable coils 14 are included on the first substrate 12 to enable pooled testing of samples. In other embodiments, even larger numbers of coils, e.g., >400 coils 14 are used to perform larger sample pooling or multiplexed analyses.

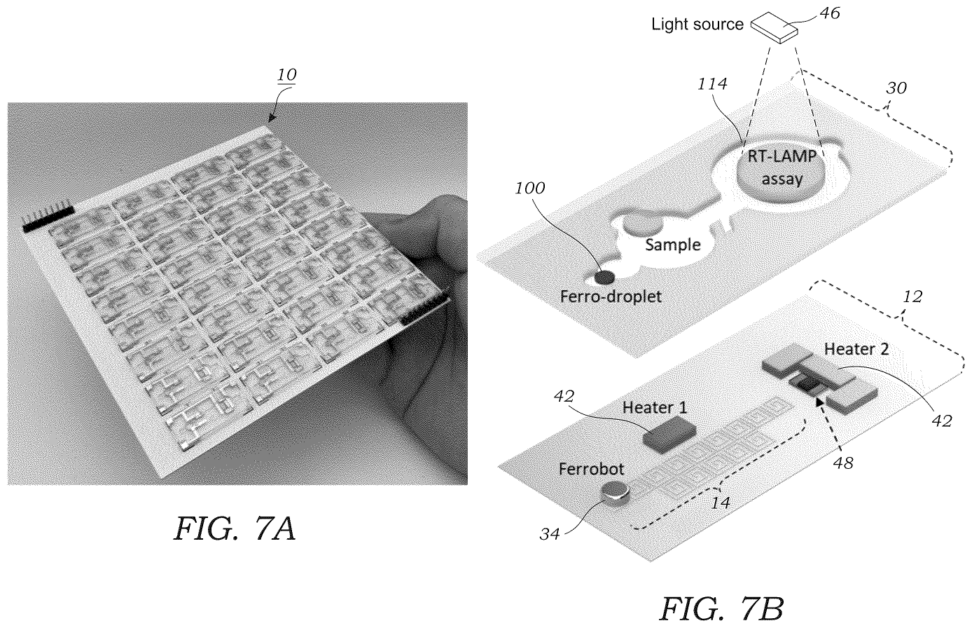

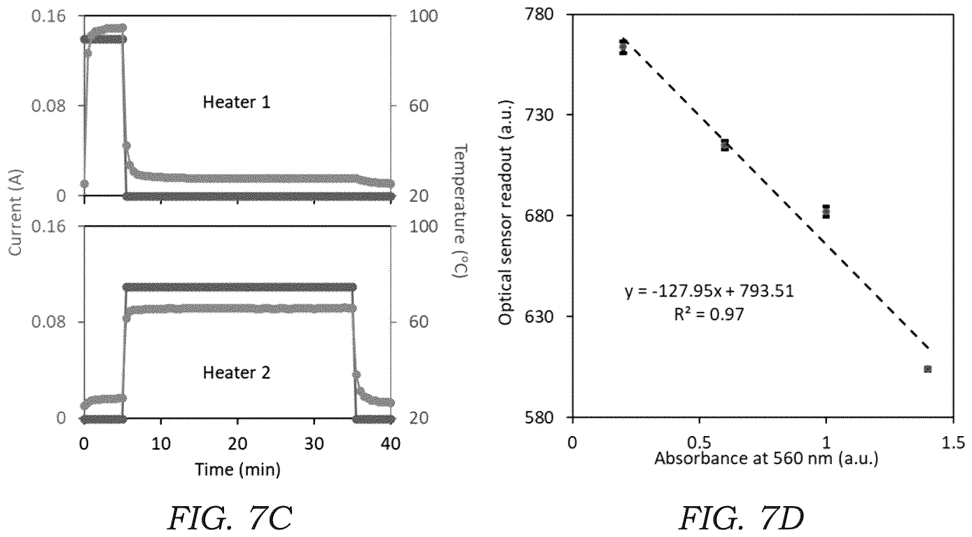

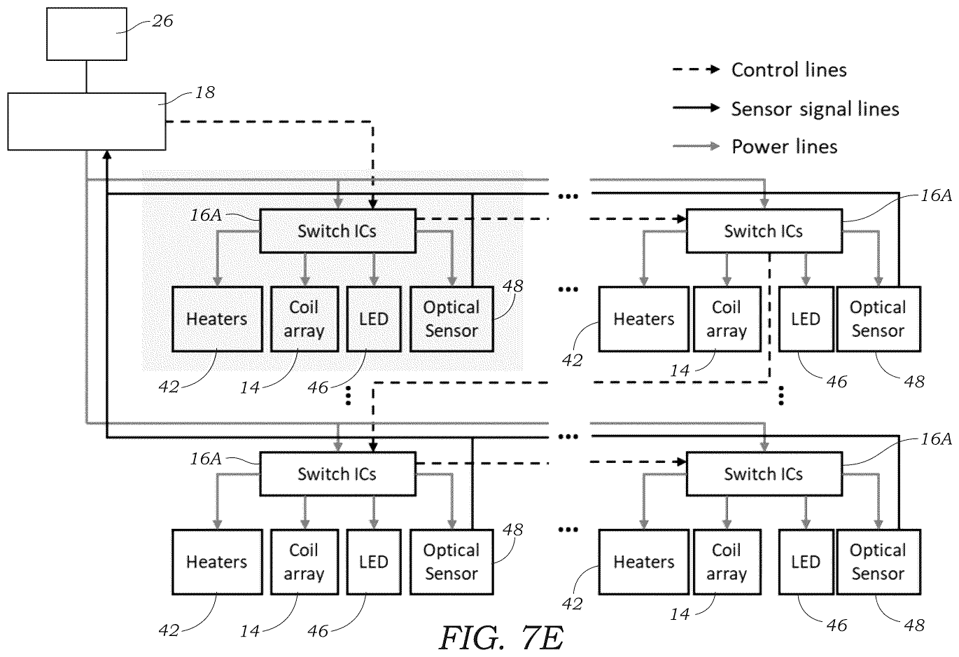

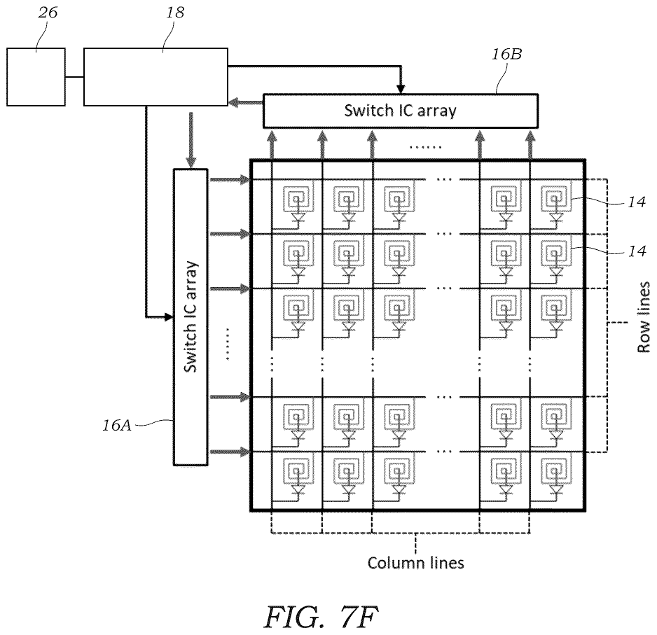

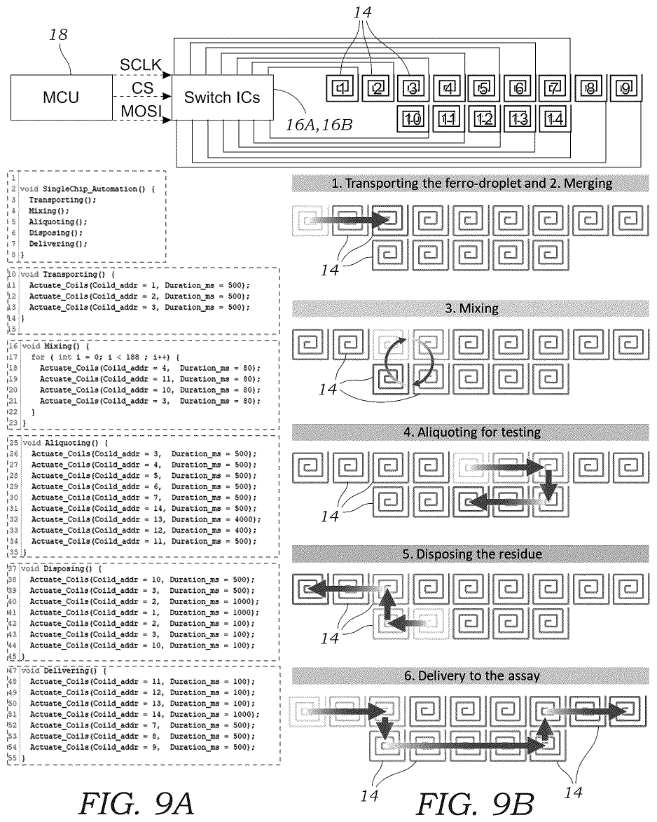

For example, in the experiments described herein, each individual addressable coil 14 had a three-turn configuration with a size of 1.5×1.5 mm stacked in three layers in the PCB making up the first substrate 12 (FIGS. 7A, 7B, 7E-7F, FIG. 9B). Adjacent coils 14 were separated by a gap of 0.1 mm. As best seen in FIGS. 1B and 7E, 7F, the first substrate 12 includes, in one embodiment, a first IC switch 16A and a second IC switch 16B. The first and second IC switches 16A, 16B may be directly integrated on the first substrate 12. The IC switches 16A, 16B are used to select and actuate or power individual addressable coils 14. For example, the first IC switch 16A may be used for row selection (e.g., MAX14662 (Maxim Integrated, CA, USA)) while the second IC switch 16B is used for column selection (e.g., MC33996 (NXP semiconductor, Netherlands).

Still referring to FIG. 1B, the first and second IC switches 16A, 16B are connected to and controlled by a microcontroller unit (MCU) 18 which acts as the control circuitry for actuating coils 14 (see also FIG. 7E). A serial peripheral interface (SPI) may connect the first and second IC switches 16A, 16B to the MCU 18. Depending on the task to be performed, and by programming at the MCU 18 level, the coils 14 can be sequentially and/or simultaneously activated to perform the desired unit operation or task as described herein. The MCU 18 may be located on-board the PCB that makes up the first substrate 12 or it may be located separate from the first substrate 12. The MCU 18 may itself be operably connected to a computing device 20 (e.g., personal computer, laptop, tablet PC, mobile phone) using, for example, a serial communication. The computing device 20 includes software 22 executed by one or more processors 24 that are used to program the sequencing and timing of actuation of the individually addressable coils 14. Target coordinates (i.e., target coils 14) are pre-programmed or sent from the computing device 20 are translated to SPI commands by the MCU 18, then transmitted to the first and second switch ICs 16A, 16B for addressable activation of the coils 14.

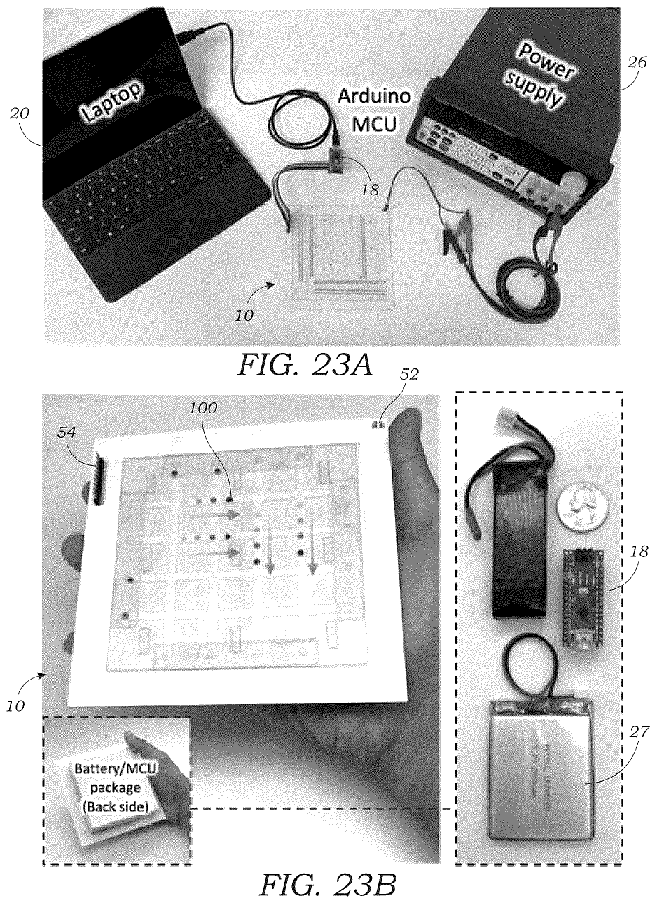

The individual addressable coils 14 are coupled to a power source through the first and second switch ICs 16A, 16B to apply a direct current (DC) to the actuated coils 14 (around 0.2 A). This may be provided using an external power supply 26. The power supply 26 may even be battery powered via a battery 27 as seen in FIG. 23B. As seen in FIG. 23B, the power supply couples to pins 52 disposed on the device 10 to provide power. The MCU 18 couples to the device 10 via interface pins 54 as seen in FIG. 23B. The software 22 may include a graphical user interface (GUI) that is used by the user to program the sequencing and timing of actuation of the individually addressable coils 14. In one aspect, the user may program the sequencing by selecting various operations that are desired to be performed. An example would be to move or transport a volume or droplet of ferrofluid 100 from point A to point B. Another example would be to create “child” or sub-volume droplets of ferrofluid 100 from a “parent” droplet (i.e., aliquot). For example, corrugated features 28 formed in the ferrofluidic fluid assay device 10 can create smaller-sized droplets (e.g., 100 nL-10 μL).

The software 22 would then automatically generate the sequence (and timing) of which coils 14 to activate to accomplish this task. In this regard, the user can easily program the desired workflow by stringing together a series of discrete operations (or sub-operations) to accomplish the desired task. Examples including, by way of illustration and not limitation, moving or transporting the one or more volumes of ferrofluid 100, forming a plurality of smaller volumes of ferrofluid 100, splitting of one or more volume of ferrofluid 100, merging one or more volumes of ferrofluid 100 with a second volume of ferrofluid 100 or a sample, mixing one or more volumes of ferrofluid 100, diluting one or more volumes of ferrofluid 100 with another fluid, removing one or more volumes of ferrofluid 100, pooling one or more volumes of ferrofluid 100, and moving ferrofluid 100 containing a sample to one or more assay chambers as described herein.

Referring to FIG. 1A, a second substrate 30 is disposed adjacent to the first substrate 12. The second substrate 30 is, in one preferred embodiment, a microfluidic chip that contains the volumes of ferrofluid 100 that are manipulated as described herein. The second substrate 30 is disposed adjacent to the first substrate 12 and separated by a gap G. Spacers 32 are optionally used to control the gap G distance. As seen in FIG. 1A, the second substrate 30 generally lies in a plane that is substantially parallel to the plane of the first substrate 12. One or more moveable permanent magnets 34 are interposed in the gap region G formed between the first substrate 12 and the second substrate 30. The permanent magnets 34 preferably comprise rare earth magnets but may also include metallic materials or composite magnetic materials (e.g., ceramic or ferrite), or other materials commonly used for permanent magnets.

The dimensions of the permanent magnets 34 may vary depending on the particular ferrofluidic fluid assay device 10 but are generally millimeter-sized permanent magnets. In experiments conducted herein, the permanent magnets 34 had a height or thickness of 0.8 mm and 2.54 mm diameter (cylindrically shaped). In some embodiments, the width or diameter of the permanent magnets 34 may be about the same or less than the width or diameter of a single coil 14. In other embodiments, the width or diameter of the permanent magnets 34 may be larger than the width or diameter of a single coil 14 thus overlapping multiple coils 14. The gap G that is formed between the first substrate 12 and the second substrate 30 is preferably kept just larger than the height or thickness of the permanent magnets 34. For example, a gap G height of around 1 mm accommodates the 0.8 mm thick permanent magnets 34, such that the permanent magnets 34 are freely moveable. As explained herein, preferably there are a plurality of permanent magnets 34 located in the gap G because each permanent magnet 34 is used to perform various tasks and unit operations. The use of multiple permanent magnets 34 allows for parallel processing of the volumes of ferrofluid 100 to take place in the second substrate or microfluidic chip 30.

The second substrate or microfluidic chip 30 contains the working area of the ferrofluidic fluid assay device 10 and contains the volumes of ferrofluid 100 where the digital operations take place. The volumes of ferrofluid 100, as explained herein, are preferably in the form of droplets 100. Preferably the droplets 100 have volumes in the range of 100 nL to 1 μL. Less preferably the droplets 100 have volumes in the range of 10 nL to 100 μL. The volumes of ferrofluid 100 contain therein magnetic particles 102. The magnetic particles 102 are preferably biocompatible and, in some embodiments, are nanoparticles. Examples of commercially available ferrofluids that include magnetic particles 102 include ferumoxytol (AMAG Pharmaceuticals, MA, USA). Some of the volumes of ferrofluid or droplets 100 also include therein a biological or chemical sample of interest that act as the “package” 104 (e.g., sample package 104) within the droplets 100. The volumes of ferrofluid or droplets 100 may also include reagents, wash solutions, indicators, and the like. The volumes of ferrofluid or droplets 100 move within the second substrate or microfluidic chip 30 in response to the strong body forces originating from the interaction of magnetic particles 102 within the volumes of ferrofluid or droplets 100 with the magnetic actuation field created by the permanent magnets 34 which are moved by the individually addressable coils 14.

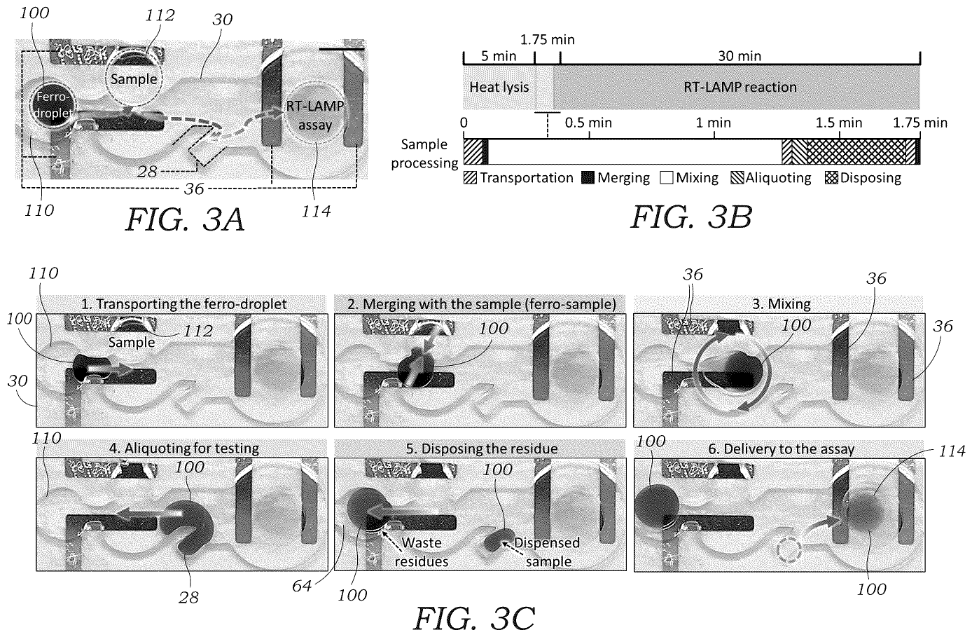

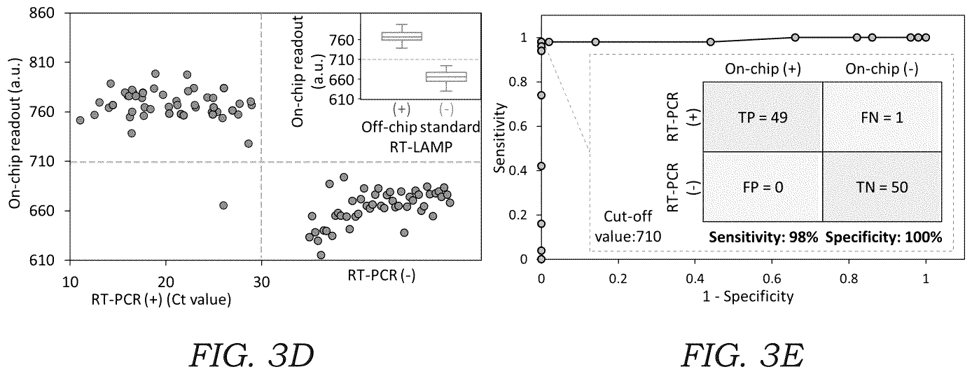

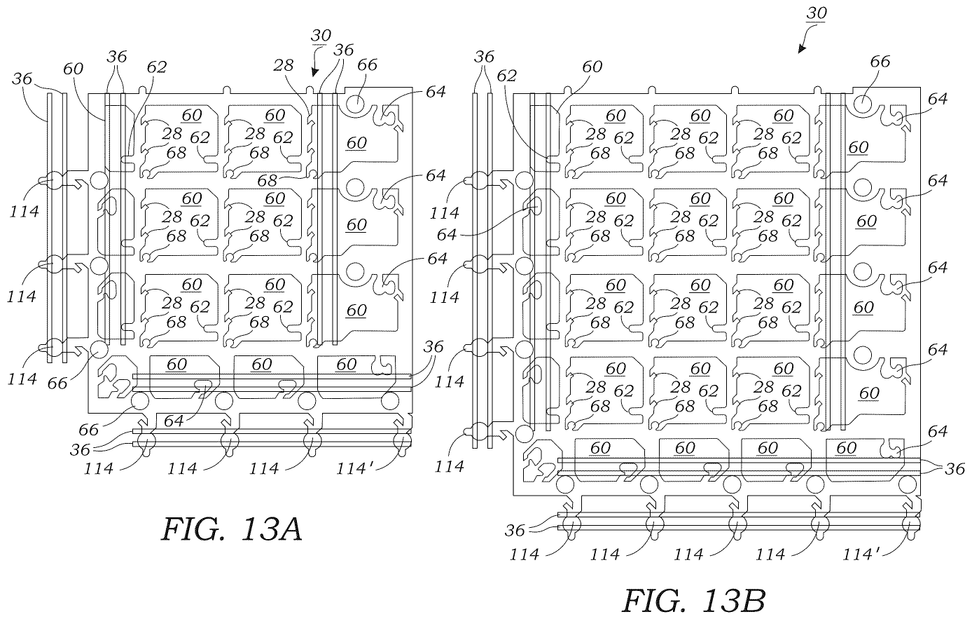

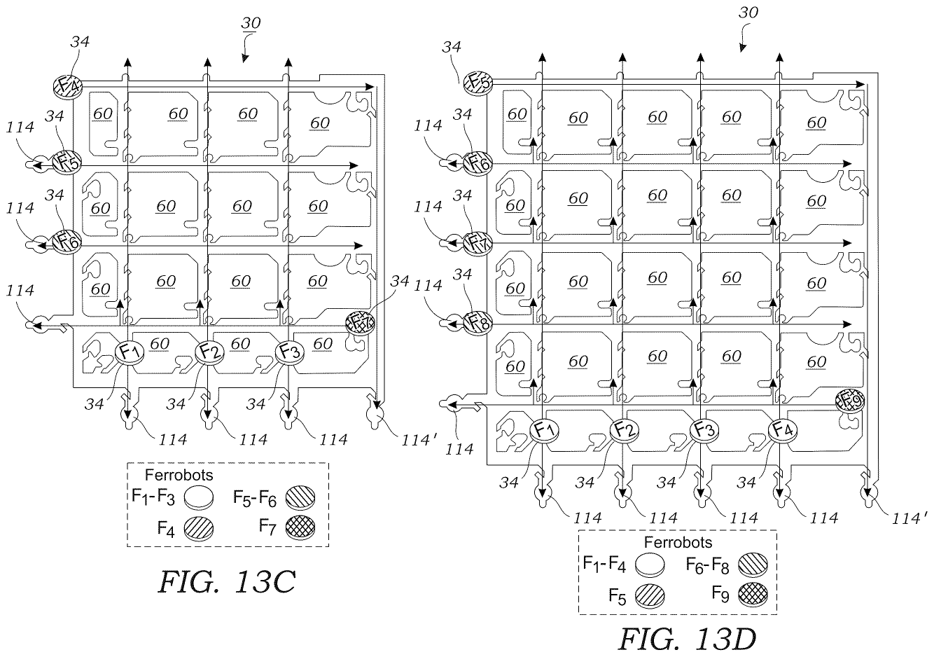

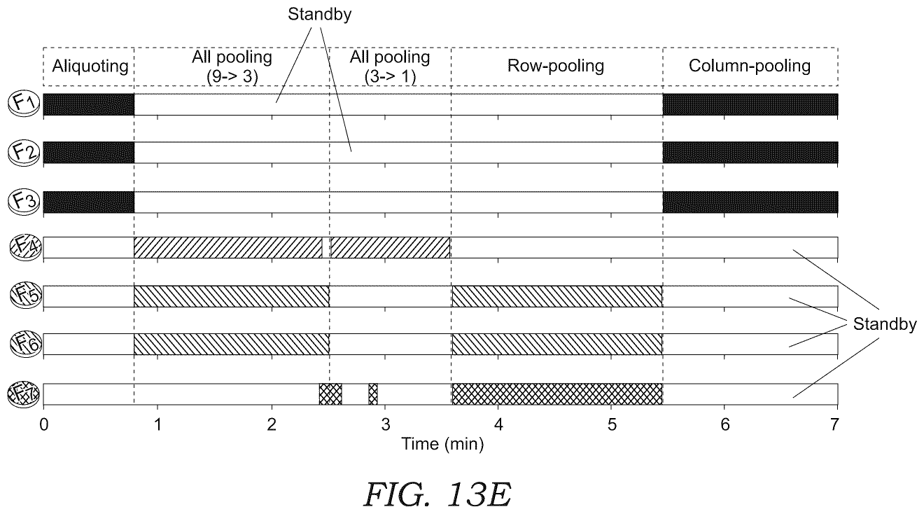

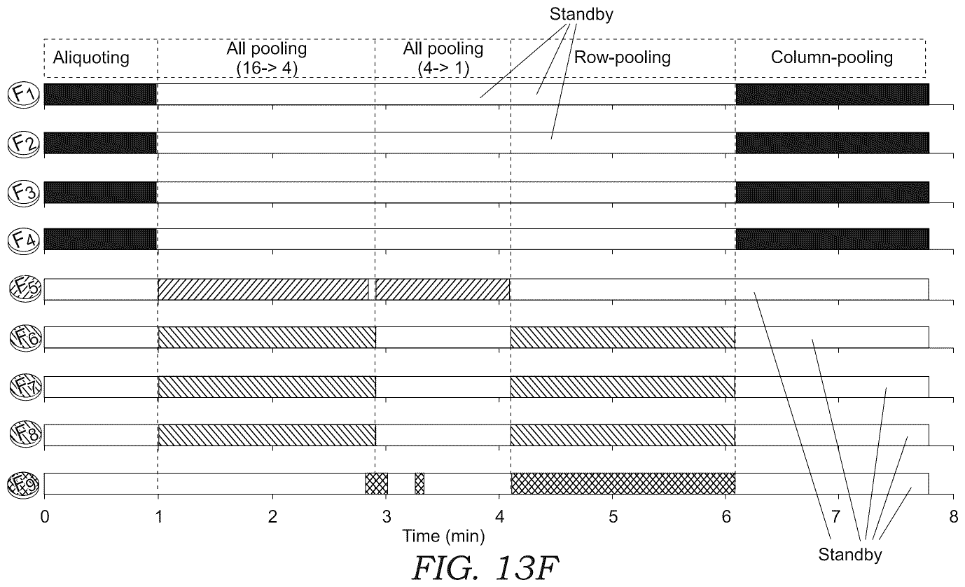

With reference to FIGS. 1A, 3A, 3C, the second substrate or microfluidic chip 30 includes one or more microfluidic features 50 thereon. The microfluidic features 50 may include microfluidic channels, walls, flow path(s), holding chambers or regions, blocks that define rows and columns that allow for movement of volumes of ferrofluid or droplets 100. As one particular example, and with reference to FIGS. 3A and 3C, these may include may include ferrofluid holding chambers 110 for holding ferrofluid to be used in the volumes of ferrofluid or droplets 100, one or more sample holding chambers or regions 112 (e.g., for loading or holding samples), one or more corrugated features 28 that are used to define and split off sub-volumes of ferrofluid 100 as they move across the corrugated structure, one or more assay chambers 114, a waste or disposal chamber 64 (e.g., FIGS. 13A-13B), a reagent chamber, and the like. In the embodiment of FIGS. 3A and 3C, the holding chamber 110 is used to hold the initial volume or droplet of ferrofluid 100 as well as hold the waste residue and operates as a disposal chamber 64 as seen in FIG. 3C. The second substrate or microfluidic chip 30 may include a central region that includes an open area that permits easy lateral (e.g., planar) travel of the volumes or droplets of ferrofluid 100 across the surface of the second substrate or microfluidic chip 30. This allows volumes or droplets of ferrofluid 100 to move between different physical locations of the second substrate 30 or microfluidic chip. For example, volumes or droplets of ferrofluid 100 can move between different physical locations where dedicated operations are performed (i.e., merging with sample, aliquot of sub-volume of sample, merging or pooling of volumes or droplets of ferrofluid 100, heating a volume or droplet of ferrofluid 100 containing a sample, cooling a volume or droplet of ferrofluid 100/sample, imaging a volume or droplet of ferrofluid 100 containing a sample, electrochemically measuring a sample, performing an assay, or waste removal).

The volumes or droplets of ferrofluid 100 are surrounded by a filler fluid 106. Typically, the volumes or droplets of ferrofluid 100 are aqueous-based and the filler fluid 106 is an oil-based filler. An example includes fluorinated oil such as Novec™ 7500 Engineered Fluid, 3M, MN, USA. An optional surfactant may also be added to the filler fluid 106 (e.g., Pico-Surf™ 1, Sphere Fluidics, NJ, USA). In some embodiments, where operations are conducted over a shorter time period or where evaporation is mitigated, an external filling fluid 106 such air or other gas may be used. In other embodiments, the volumes or droplet of ferrofluid 100 may be oil-based with the filler fluid 106 being an aqueous-based filler.

The second substrate or microfluidic chip 30 may be formed as a laminate structure that is formed by multiple layers 56 of a polymer that are adhered to each other using an adhesive or tape 58 with adhesive backing. For example, polyethylene terephthalate (PET) film sheets may be used with double-sided tape to form the laminate structure. Additional materials such as plastics or polymer materials or glass may be used with manufacturing processes known in the art, such as hot embossing, injection molding, 3D printing and the like. The physical features on the second substrate or microfluidic chip 30 can be created using laser-cutting. In some embodiments, electrodes 36 may be deposited or patterned prior to assembly. The second substrate or microfluidic chip 30 includes a top surface 38 and a bottom surface 40. The bottom surface 40 typically does not have any openings therein as it forms the floor on which the volumes or droplets of ferrofluid 100 move. The top surface 38 may be closed, open, or partially open. For example, openings in the top surface 38 may be used to deposit fluid samples and/or reagents into the second substrate or microfluidic chip 30. Likewise, openings may be used to remove fluid from the ferrofluidic fluid assay device 10.

FIGS. 1G-1I illustrates a representative ferrobotic testing platform, which includes two modules (entirely constructed by low-cost components): 1) a disposable oil-filled microfluidic chip 30 with passive and active actuation interfaces that hosts input sample(s) and ferrofluid/assay reagents and 2) a printed circuit board (PCB) as the first substrate 12, featuring 2D arrayed coils 14 (“navigation floor”), which can be independently activated to electromagnetically direct individual ferrobots 34.

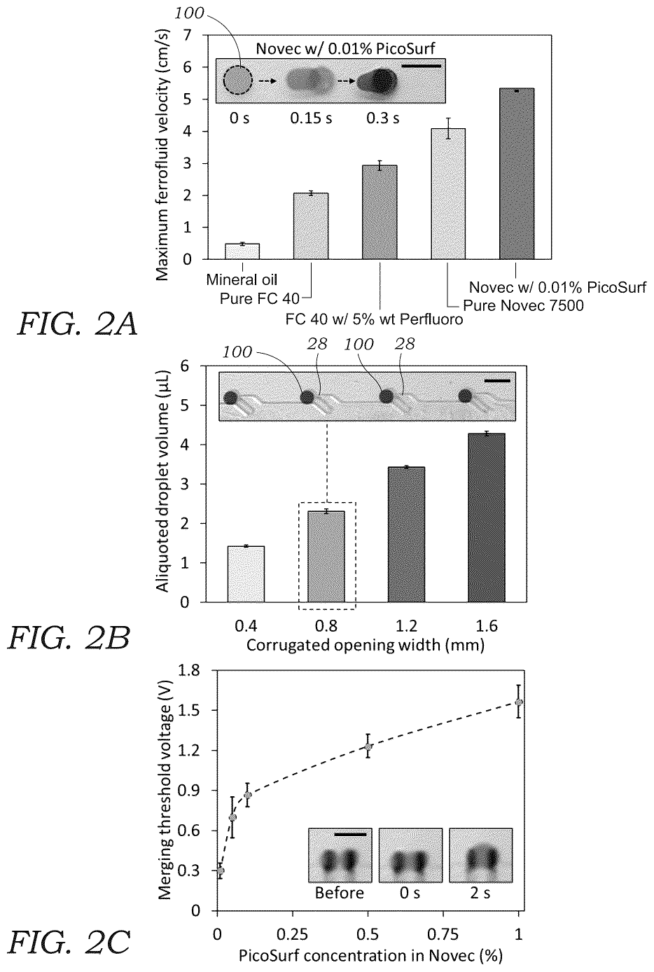

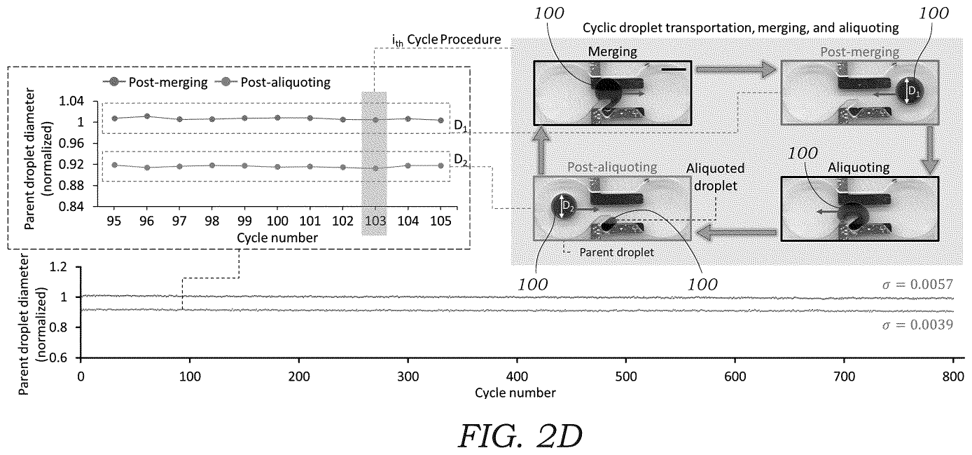

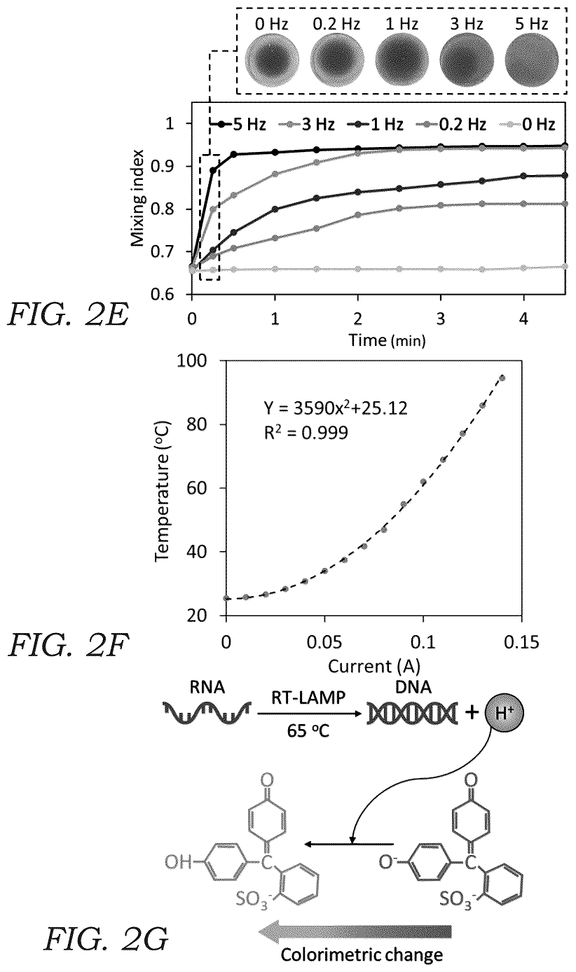

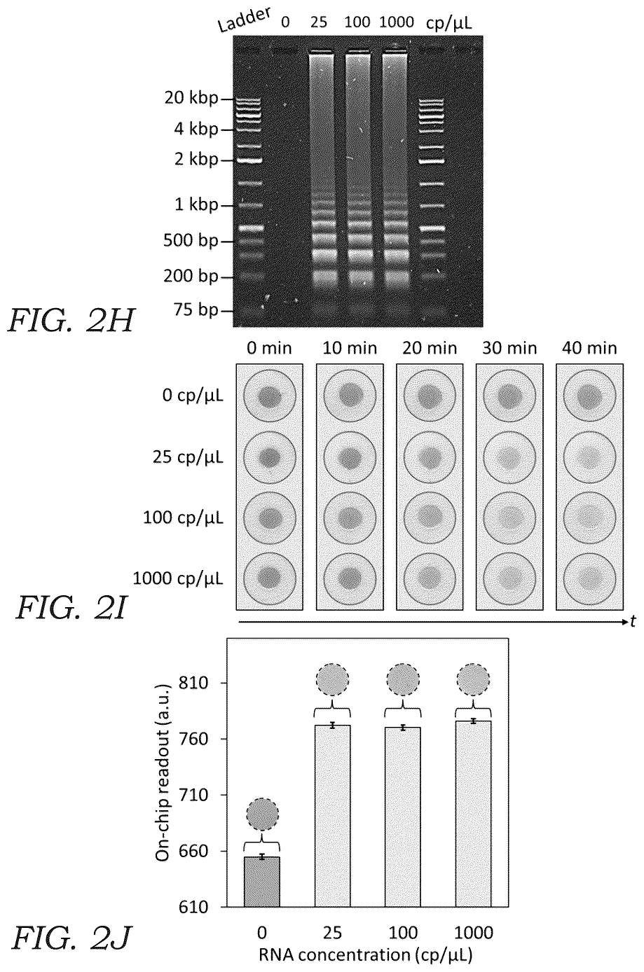

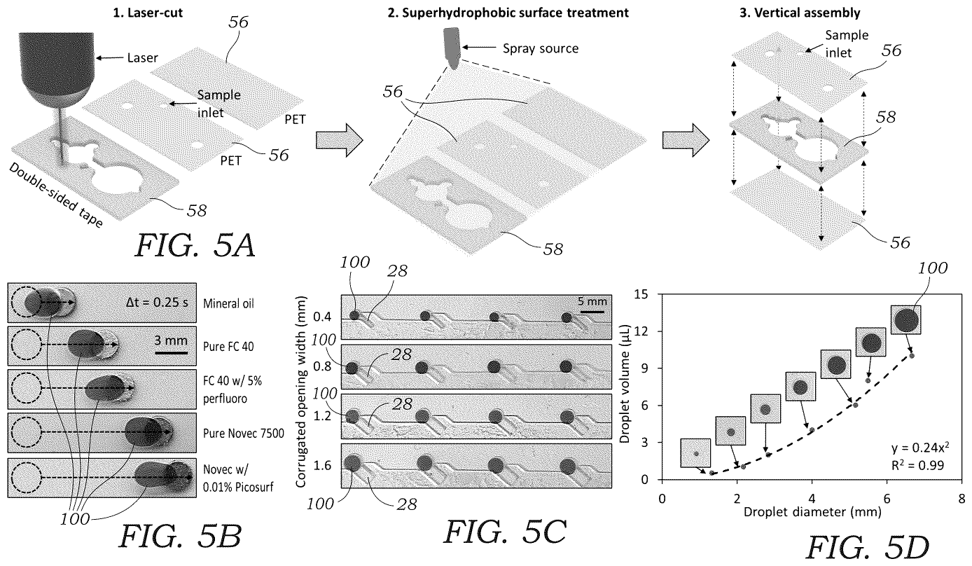

The miniaturized bioanalytical operations and workflows were realized with the digital ferrofluidic fluid assay device 10, because it simultaneously offers high degrees of robustness, diversity, programmability, and scalability for low-volume sample handling. Within this framework, a suite of operations was developed and characterized, including droplet transportation, aliquoting, merging, mixing, and heating, which are key to the on-chip implementation of NAAT-based assays (FIGS. 2A-2J, FIGS. 5A-5D).

By programming the underlying PCB-based coils 14, one can electromagnetically direct the ferrobots 34 to carry volumes or droplets of ferrofluid 100 within different oil environments, where rapid droplet transportation with a maximum velocity range of 5-50 mm/s was achieved (FIG. 2A, FIG. 5B). It was found that Novec™ (oil)/PicoSurf™ (surfactant) yielded the maximum speed of the volumes or droplets of ferrofluid 100 (owing to its lower viscosity), while being compatible with the RT-LAMP assay.

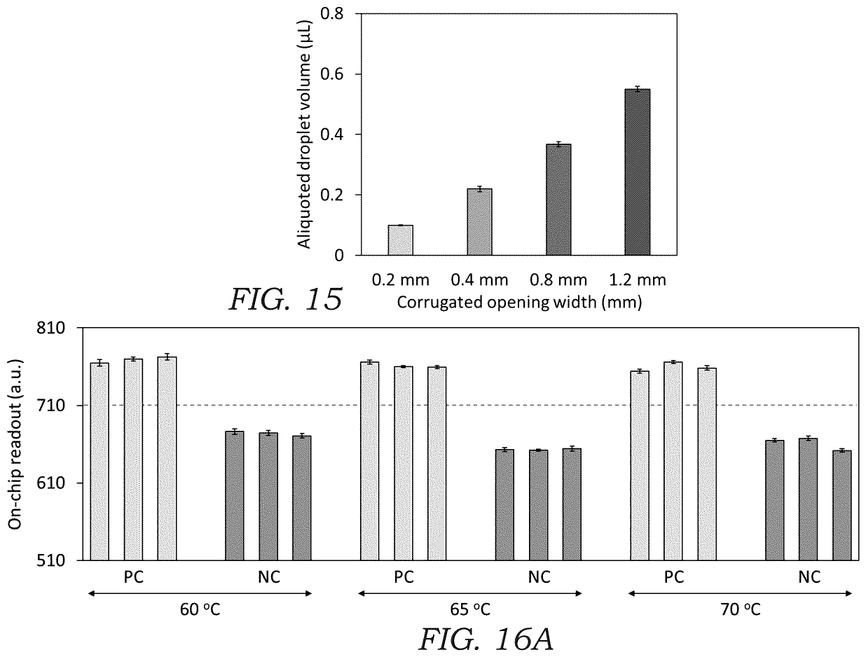

FIG. 2B illustrates the precise and tunable ferrobotic sample aliquoting capability in the optimized Novec™ oil environment. In this context, aliquoting is a critical step for precise sample metering and creating sub-volumes for multiplexing and multi-round pooling analysis. Aliquoting is achieved by directing a volume or droplet of ferrofluid 100 carried by the ferrobot permanent magnet 34 along a corrugated structural feature 28, which in turn causes the dispensing of a smaller volume or droplet of ferrofluid 100 (as an aliquot). By adjusting the opening of the corrugated feature 28 and/or the channel height, the volume of the aliquot can be tuned over two orders of magnitude (e.g., here 100 nL-10 μL; FIGS. 5C, 5D and FIG. 15).

To realize merging of the volumes or droplets of ferrofluid 100, the principle of electrocoalescence was used. In this context, droplet merging is useful for adding reagent(s) to the input sample(s) and combining multiple input samples for pooling. As shown in FIG. 2C, by transporting the volumes or droplets of ferrofluid 100 to a pair of electrodes 36 and applying a relatively low voltage (~0.3 V-1.5 V, depending on the surrounding oil surfactant composition) droplet merging in less than a few seconds can be achieved. Droplet 100 merging can also be achieved without application of voltage by bringing droplets 100 in contact to each other using a ferrobot magnet 34 in a surrounding environment without surfactant.

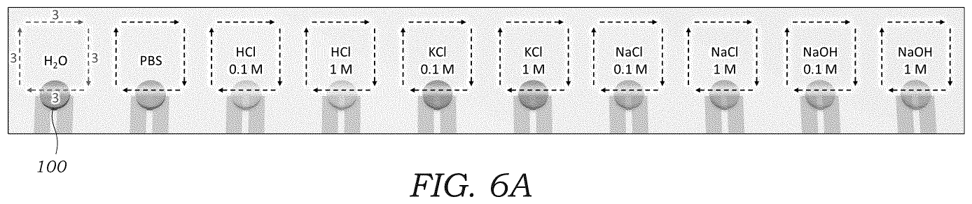

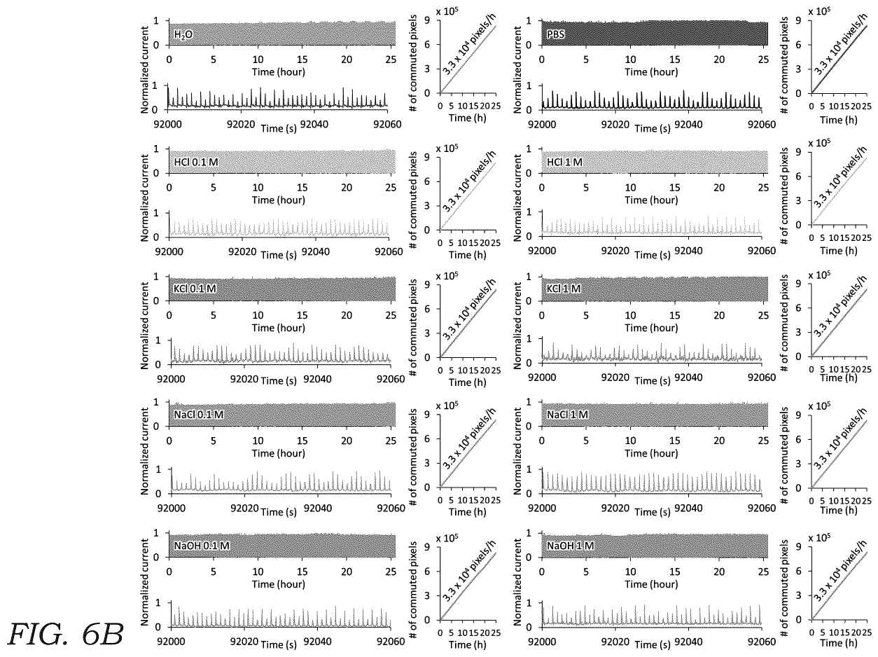

It was found that robust and repeatable ferrobotic droplet actuation can be achieved for volumes or droplets of ferrofluid 100 spanning different ionic strengths and chemical compositions relevant for biological and chemical assays (FIGS. 6A-6B). A total of >8 million actuation events were performed over >24 h (only limited by the observation time) showing repeatable behavior over the time period. This behavior differs from common digital microfluidics approaches such as electrowetting on dielectric (EWOD), which undergo surface degradation-related issues. Further illustrating that other ferrobotic operations are robust, cyclic aliquoting, merging, and intermediate transportation of a parent volume or droplet of ferrofluid 100 over 800 cycles was performed with <1% variation in the corresponding size of the parent droplet post-aliquoting and post-merging (FIG. 2D).

To realize mixing, which is particularly important for homogenizing the droplet contents post-merging, the permanent magnet 34 can be oscillated to induce chaotic fluid motion within the merged volume or droplet of ferrofluid 100 by alternatively activating the neighboring coils. As shown in FIG. 2E, the droplet homogenization rate increases with oscillation frequency, and in particular, a nearly full-mixed state can be reached in ~15 seconds by oscillating the ferrobot 34 at 5 Hz.

Optional, on-board resistive heaters 42 (FIG. 7B) were used for nucleic acid amplification and sample preparation (e.g., lysis). The local temperature can be controlled by adjusting the DC current flowing through the resistive heater, in accordance with the operational needs (FIG. 2F, FIGS. 7A-7C). The heater(s) 42 may be disposed on the first substrate 12 or the second substrate 30.

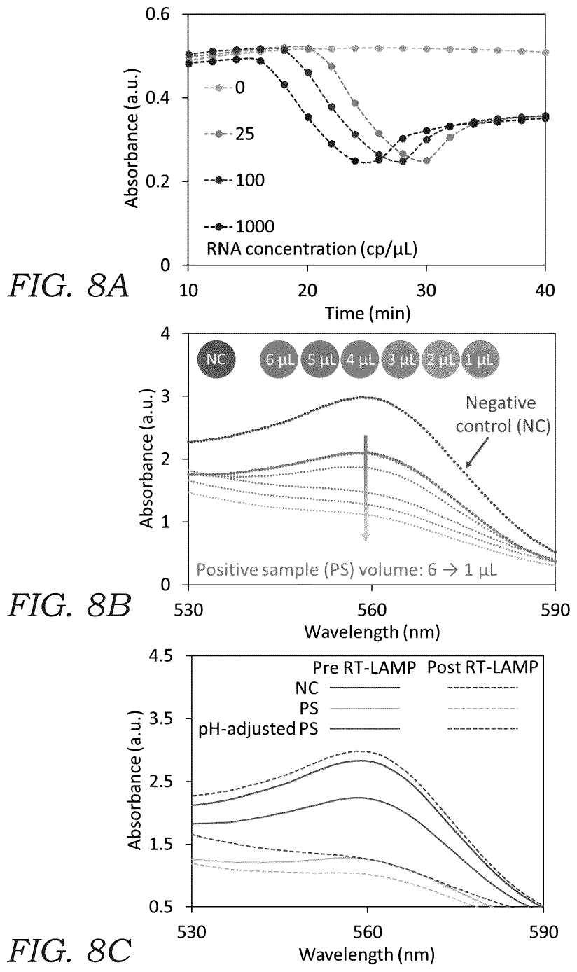

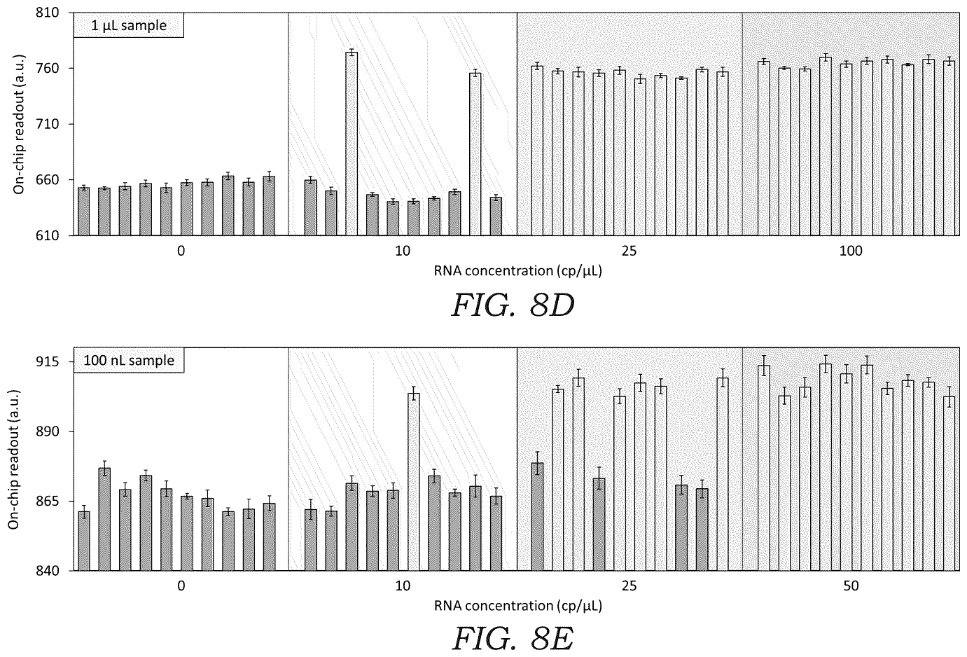

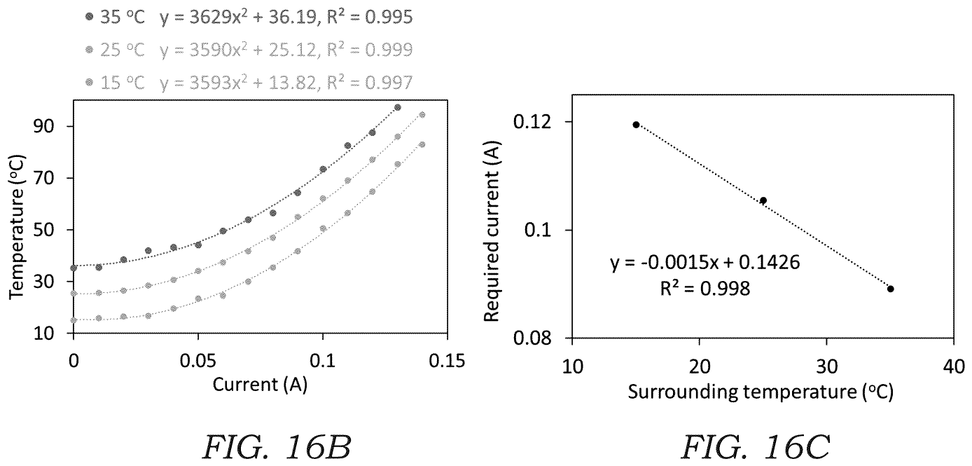

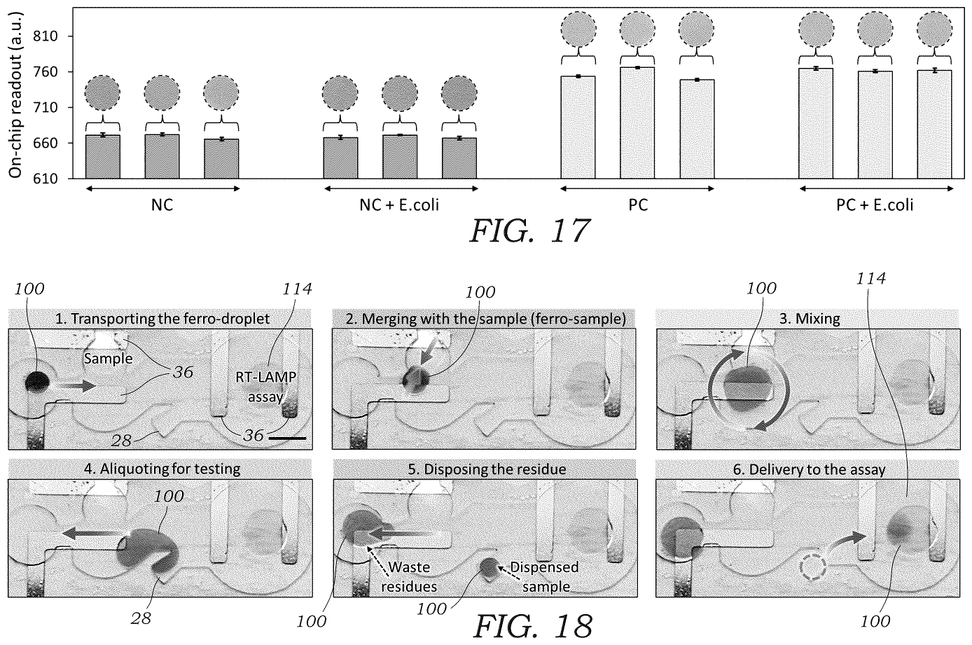

A colorimetric RT-LAMP assay was implemented that is based on thermal lysis/inactivation and isothermal amplification (both achievable with on-board resistive heaters). This assay provides a high degree of test accessibility, outweighing the marginal compromise in test accuracy. FIG. 2G illustrates the RT-LAMP reactions, which involve the reverse transcription of the viral RNA, amplification of the product DNA, and generation of hydrogen ions, which are colorimetrically detected in an assay chamber 114. By analyzing the reaction product (DNA) via gel electrophoresis (FIG. 2H), the assay function was verified in converting and amplifying a severe acute respiratory syndrome coronavirus 2 (SARS-CoV-2) positive control RNA sample. Colorimetric detection is based on the generated hydrogen ions, causing a color change of an incorporated pH indicator (phenol red) from red-orange to yellow (optimization experiment results shown in FIGS. 8A-8C). The color change allows for the binary interpretation of the test, above or below a threshold as positive or negative, respectively. This color change can be optically interrogated visually (FIG. 2I) by the naked eye, or electronically by integrating the response of an optical sensing module 44 (FIG. 2J and FIGS. 7B, 7D), without the absorbance of the ferrofluid affecting the readout interpretations. The optical sensing module 44 includes a light source 46 and a light or optical sensor 48. Similarly, an electrochemical pH sensor (not shown) may be used to quantitatively readout the result of the assay. Accordingly, the same limit of detection of 25 cp/μL of the adopted assay can be achieved in the ferro-droplet format (1 μL; reagent volume 19 μL; similar to the original assay protocol), suggesting the magnetic nanoparticles 102 do not interfere with the amplification chemistry or colorimetric readout accuracy (FIG. 8D). The assay was also successfully performed by using microfluidic structures of reduced height (~150 μm) to aliquot a 10-fold smaller ferro-droplet volume (100 nL; reagent volume 1.9 μL; FIG. 8E), which is below the volume that can be accurately pipetted using robotic liquid handlers, but useful for minimizing reagent use. The characterization results also verified the reliability of the assay in the presence of temperature variations of a few degrees c° (FIG. 16A) and in the presence of biological interferents (FIG. 17).

The programmability of the ferrofluidic fluid assay device 10 (FIGS. 9A-9B) allows for its ease of adaptation to streamline the ferrobotic actuation and bioanalytical operations and deliver versatile RT-LAMP-based testing workflows in an entirely automated fashion and with high fidelity. It should be appreciated that other nucleic assay amplification formats may also be conducted in the ferrofluidic fluid assay device 10 (e.g., RT-PCR, etc.).

Illustrating this point in the context of individual sample testing, a disposable microfluidic chip including the first substrate 12, second substrate 30, permanent magnets 34, addressable coils 14 and other features of the device 10 described herein was customized to host the input sample, associated reagents, and dedicated aliquoting/merging components (FIG. 3A, FIG. 7B). A PCB module was used as the first substrate 12 containing the navigation coils 14, resistive heaters 42, and colorimetric sensing circuitry. By programming the PCB 12 at the software-level, a ferrobotic instruction set was installed to seamlessly execute the assay. The instruction set charts the navigation plan of a dedicated permanent magnet 34 “ferrobot” and details the electrode 36 excitation conditions for merging and heating, while accounting for a 5 min-heat lysis and a 30-min RT-LAMP reaction period (FIG. 3B).

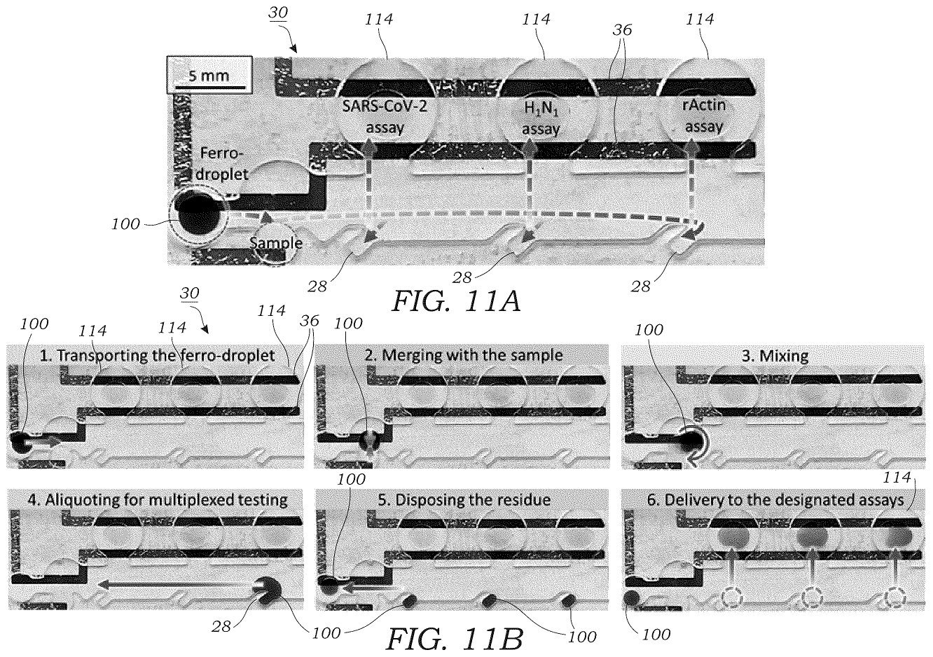

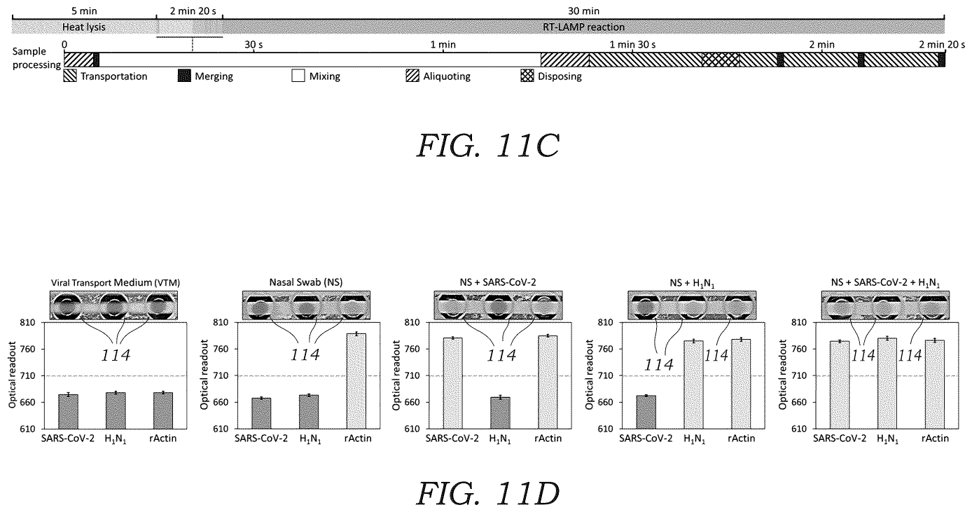

In this testing workflow, the active ferrobotic operations take place over a period of 1.75 minutes (FIGS. 3B-3C). A volume or droplet of ferrofluid 100 is first magnetically transported to, then merged and mixed with an introduced sample droplet, in order to make the sample amenable for ferrobotic manipulation. The next steps in the sequence are aliquoting the ferro-sample using, for example, the corrugated features 28, disposing the ferro-sample residue, and delivering the aliquot (1 μL) to the assay chamber 114 (containing the assay reagents). Upon delivery to the assay chamber 114, the RT-LAMP process initiates, and after 30 min, the assay readout is colorimetrically quantified, rendering the test result in a sample-to-answer manner (FIGS. 11A-11B). A similar workflow was implemented using microfluidic chips with reduced height (in second substrate 30) to achieve smaller ferro-droplets (~100 nL) for analysis with reduced reagents (FIG. 18).

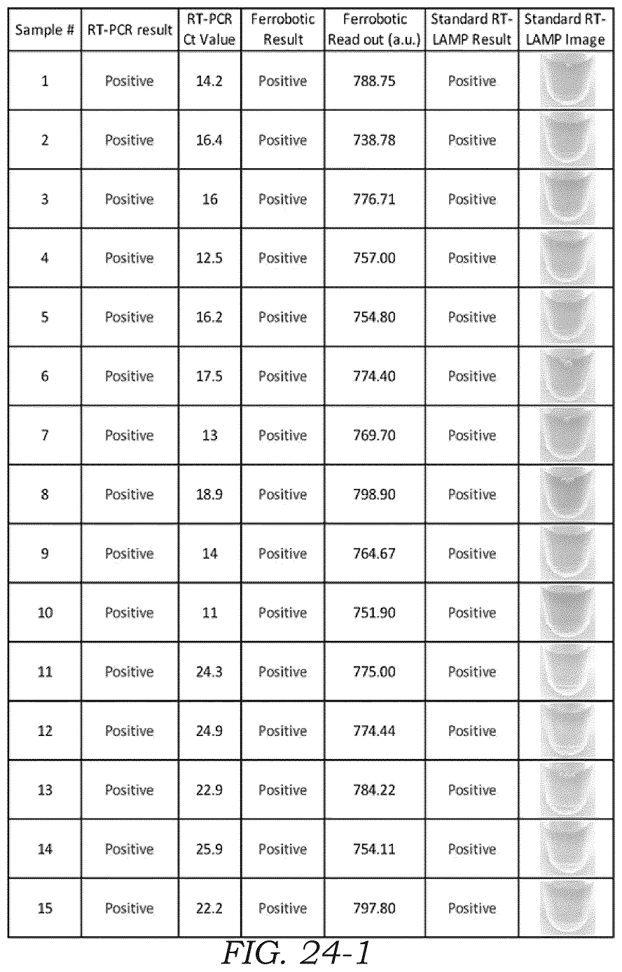

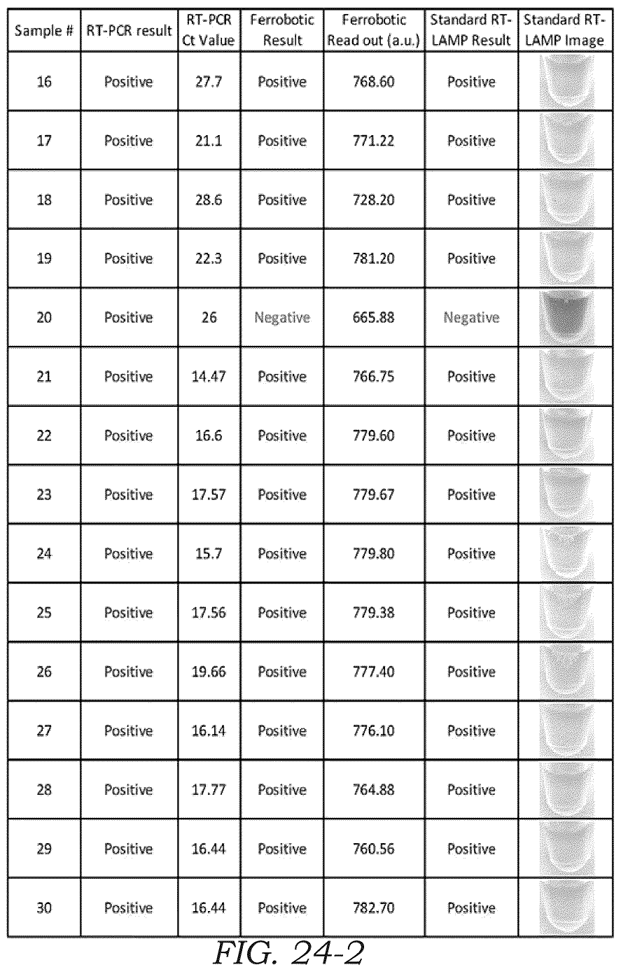

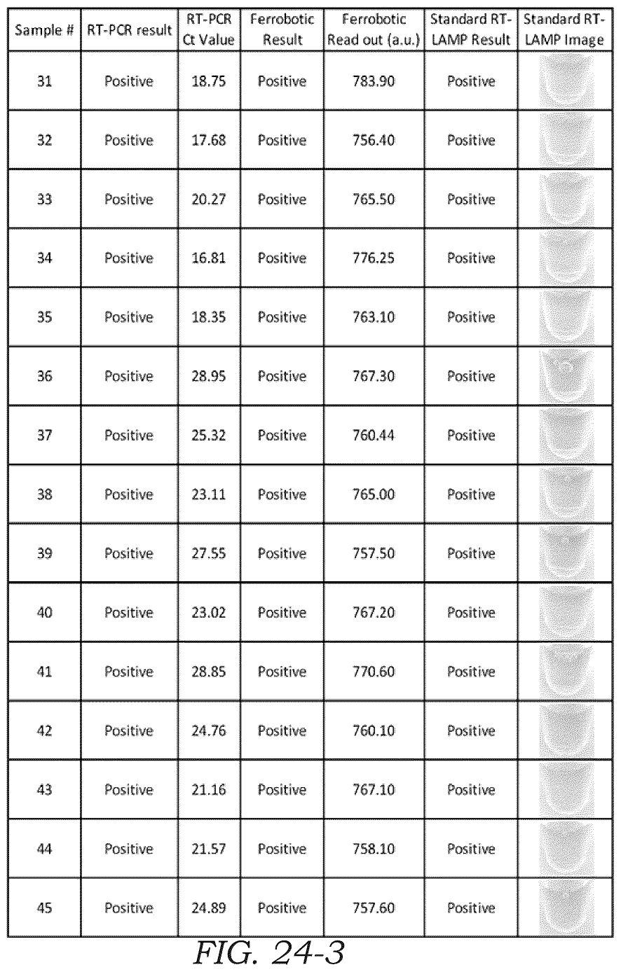

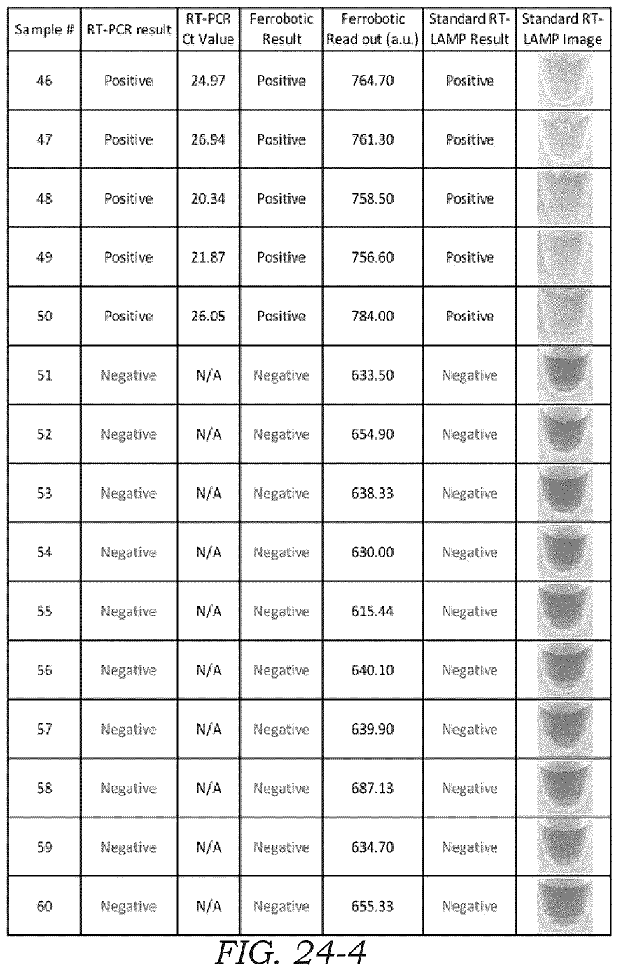

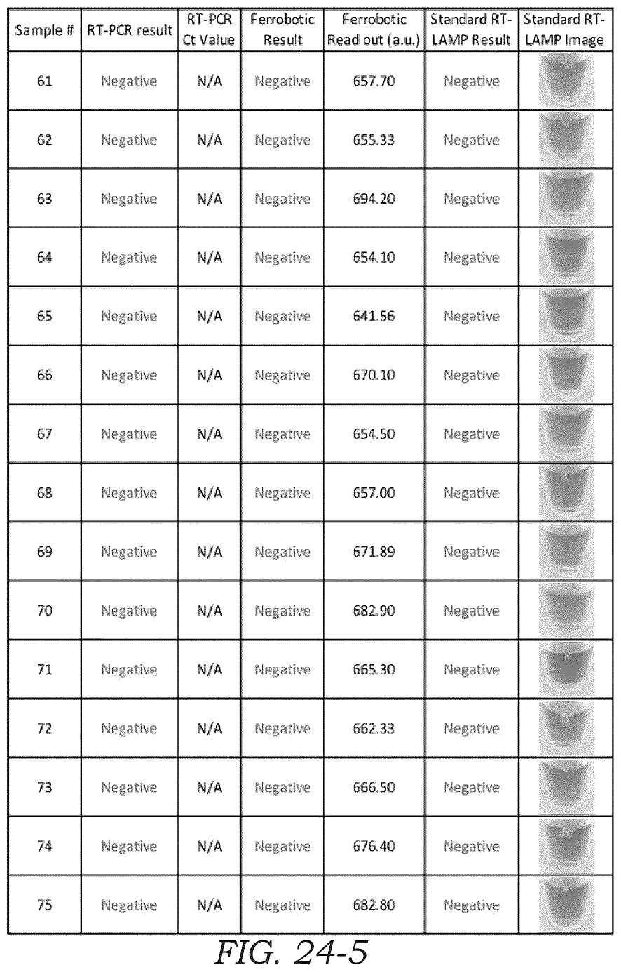

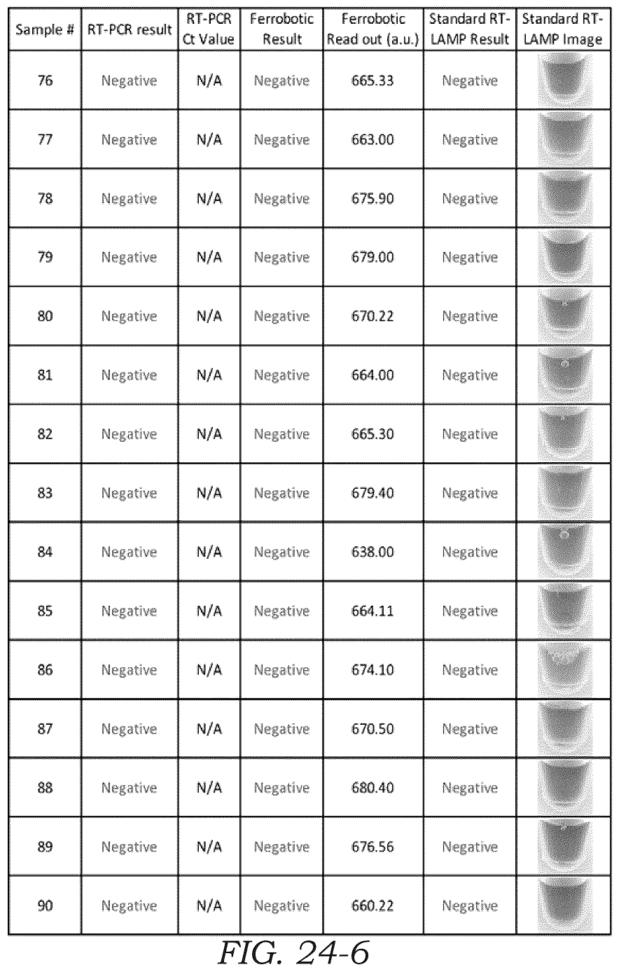

The accuracy of the device 10 was assessed with real world samples by testing one hundred clinical samples with the ferrobotic RT-LAMP chip, and comparing the on-chip readouts with the corresponding readouts obtained from the standard RT-PCR and RT-LAMP assays (summarized in FIG. 3D, detailed in FIGS. 24-1 through 24-6). The collected samples were based on nasopharyngeal swabs from SARS-COV-2 infected or uninfected patients. The viral on-chip detection threshold (710 a.u.) was derived from receiver operating characteristic (ROC) analysis (aliquoted sample volume: 1 μL).

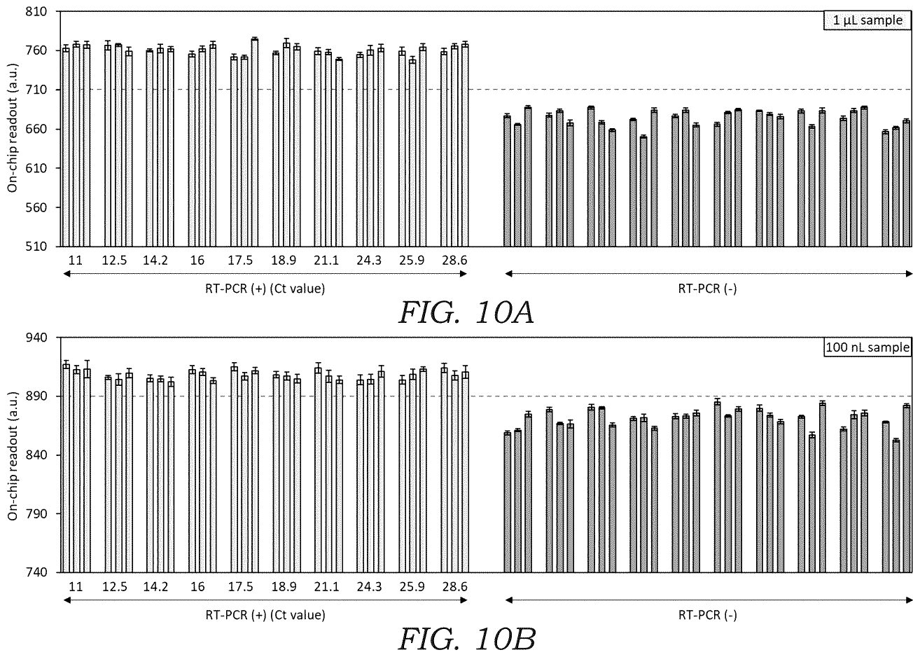

For all one hundred samples, the ferrobotically produced results were in agreement with the manually performed (off-chip) RT-LAMP assay results (100% concordance), illustrating the high fidelity of the ferrobotic automation. Comparison of the ferrobotically produced RT-LAMP-based results with the corresponding results obtained from the RT-PCR assay (gold standard) resulted in a test sensitivity of 98% and specificity of 100% (FIG. 3E), wherein the rare test result discrepancy can be attributed to the inherent differences of the employed amplification approaches. It was further validated that the clinical samples with aliquoted volumes of 1 μL and 100 nL can be accurately analyzed in a reproducible manner across replicates (FIGS. 10A-10B).

Multiplexed viral testing was demonstrated by leveraging the platform's adaptability (FIGS. 11A-11D). This testing mode is diagnostically useful for differentiating between the emergent outbreak virus (e.g., SARS-COV-2) and endemic viruses (e.g., the seasonal ones such as influenza A (H1N1) pdm09 virus) that often result in similar clinical symptoms.

By capitalizing on the platform's scalability, one can increase the testing throughput. The extensibility of the employed mobile robotic scheme to a multi-agent mobile (swarm) robotic scheme, together with the expandability of the navigation floor/microfluidic architecture, inherently render the device 10 scalable. One approach to increasing the throughput is to simply extend the individual testing platform into an array format (FIG. 7A, 7E). With this implementation, a large number of input samples can be analyzed in parallel and asynchronously as they arrive without involving accumulation wait time (unlike the case for current high throughput methods that rely on batch-processing). A less trivial, yet more efficient high throughput testing approach involves applying the device 10 to the problem of adaptive pooled testing.

To determine the appropriate number of input samples and guide the pooled testing workflow, the prevalence-based adaptive testing algorithm was leveraged that can be implemented following a square matrix pooling scheme (FIGS. 1C-1E). Following this approach, testing efficiency can be substantially improved in moderate-to-low viral prevalence ranges (specifically, by appropriately performing 3² or 4² matrix pooling, determined algorithmically).

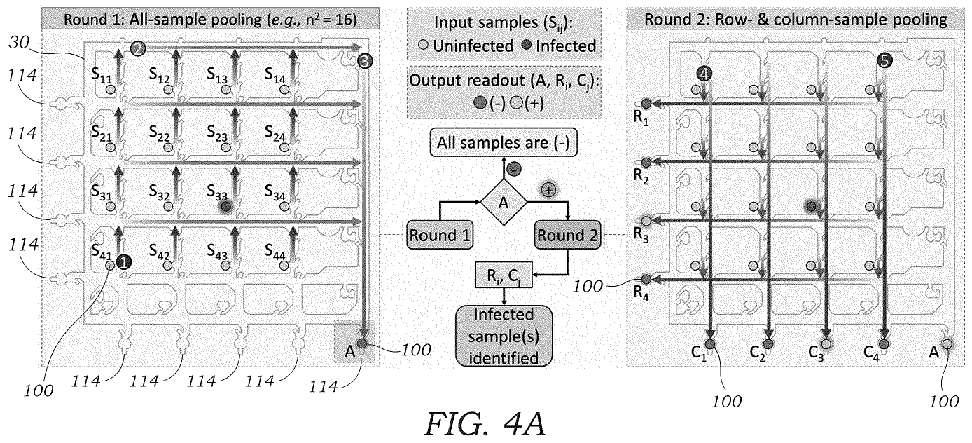

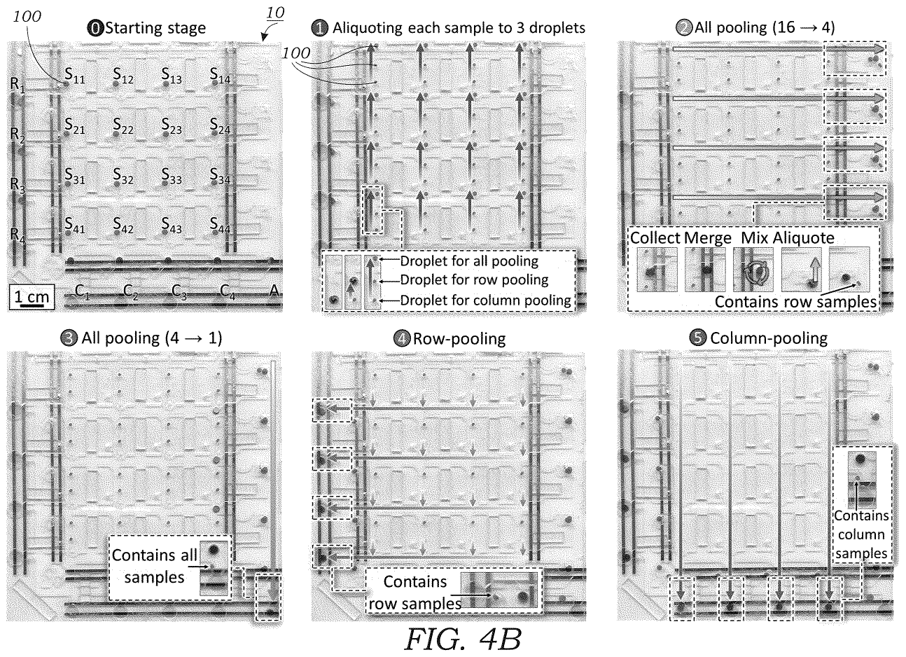

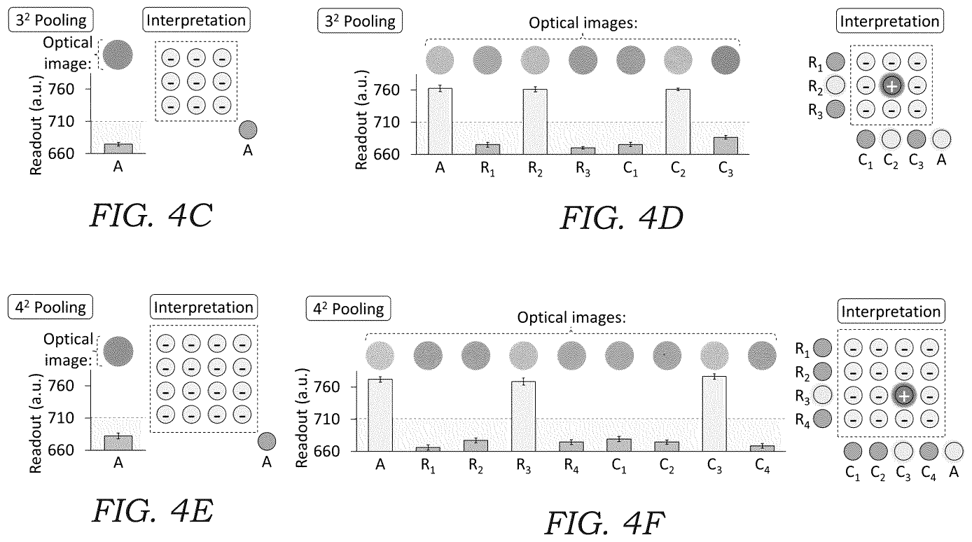

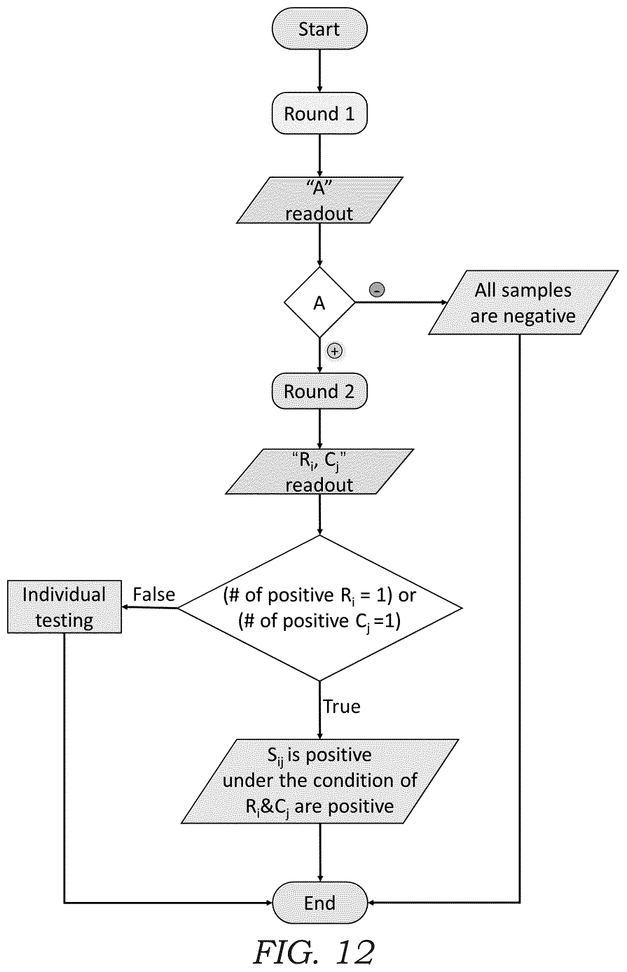

FIG. 4A provides an overview of the algorithm-guided square matrix pooling scheme, particularly for the case of 4² pooling, which involves a group of 16 samples arranged in a 4×4 matrix (Sᵢⱼ, i,j represent the row and column indices, respectively). In this scheme, all the samples are first pooled together and the resultant sample aggregate will be analyzed by a single assay “A”. If the assay readout is negative, all the original input samples will be deemed negative. Otherwise, a second round of testing will be followed. In this round, the samples will be pooled along rows and columns, leading to a total of eight sample aggregates. The row-pooled and column-pooled sample aggregates will be correspondingly analyzed by dedicated “Rᵢ” and “Cⱼ” assays. The intersectional analysis of the Rᵢ and Cⱼ assay readouts allows for determining the infected sample(s) (FIG. 12 and FIG. 1F). In the relatively low probable cases (e.g., 2.5%, assuming the viral prevalence of 2%) where the paired row/column projections are not one-to-one mapped to specific arrangements of multiple positive samples, only those samples that are deemed suspicious (i.e., those located at the intersection of positive row/column projections) will be individually tested.

To implement the square matrix pooled testing workflow, the microfluidic chip layout was expanded for pooled testing. FIGS. 13A, 13B illustrates the corresponding layouts of the 3² and 4² microfluidic chips. The expanded layouts especially include arrays of sample aliquoting interfaces using corrugated features 28 and assay chambers 114 (containing SARS-COV-2 RT-LAMP assay solutions), orthogonal corridors for intra-chip sample aliquot transport, and extended merging interfaces. In one embodiment, the microfluidic chip 30 contains a series of blocks 60 that define columnar and row flow paths within the microfluidic chip 10. The perimeter of the blocks 60 contain structural features to perform one or more operations. For example, aliquoting structures in the form of a corrugated surface 28 is positioned on one side of the blocks 60. Sample inlets 62 are located on another surface of the block 60 where sample is loaded into the microfluidic chip 30. Sample inlets 62 may be located adjacent to sample holding regions 112 although sample holding regions 112 may also be located in areas away from the sample inlets 62. Disposal chambers 64 are also located on surfaces of some of the blocks 60 which act as chambers to store waste or unwanted fluid(s). Mixing regions 66 are located along the rows and columns defined by the blocks 60 and are used to mix the ferrofluid volumes or droplets 100. Droplet holders 68 are provided on a surface of the blocks 60 and are used to hold droplets of ferrofluid 100 and/or sample. Assay chambers 114 are located at the ends of the rows and columns as illustrated in FIG. 13A. One assay chamber 114′ is located at the edge of the microfluidic chip 30 and is used to perform analysis on the pooled sample from all samples. The remaining assay chambers 114 are used to perform analysis on their associated column or row. Two sets of electrodes 36 are present at the ends of the rows and columns for the row and column electrocoalescence of volumes or droplets of ferrofluid 100 to achieve pooling. To direct the swarm ferrobotic operations in accordance with the devised pooling scheme, a PCB module 12 was utilized with increased navigation coils 14 (i.e., an expanded navigation floor) and programmed the PCB module 12 to install an updated multi-ferrobot-based and pooling algorithm-driven instruction set. FIG. 13B illustrates a similar microfluidic chip 30 as disclosed in FIG. 13A with the difference that rather than a 3×3 construction, a 4×4 construction is used for holding 16 samples. Additional N×N sample pooling follows a similar structure except for N rows and N columns and N2 samples (here N is a number greater than 4). The footprint of the device increased geometrically, but the construction otherwise remains the same.

FIG. 4B illustrates the sequence of the operations performed by a swarm of nine permanent magnets 34 or ferrobots to deliver a representative 4² pooled testing workflow (FIG. 13B also illustrates the nine permanent magnets 34 used in the swarm operations). The demonstrated sequence involves: 1) making three aliquots of each input sample with the aid of four permanent magnets 34 or ferrobots. Each aliquot may be made by a separate corrugated feature 28 which can be made to generate the smaller volumes or droplets of ferrofluid and sample 100; 2) all-sample pooling to facilitate the first round of testing (performed in two steps; combining the aliquots on the same row using four ferrobots 34 in parallel, followed by combining the resultant aggregates using a single ferrobot 34); and 3) row/column pooling to facilitate the second round of testing (each performed by a set of four ferrobots 34). To combine the intended aliquots in each of the pooling steps, the aliquots were ferrobotically collected, merged, mixed, and then dispensed as a droplet with a metered volume (1 μL). The final volume may be delivered to an assay chamber 114 for visualization as explained herein. The overview of the navigation plan and the detailed timeline of the task sequence executed by each ferrobot 34 (in coordination with the other ferrobots 34) are illustrated in FIGS. 13C-13F.

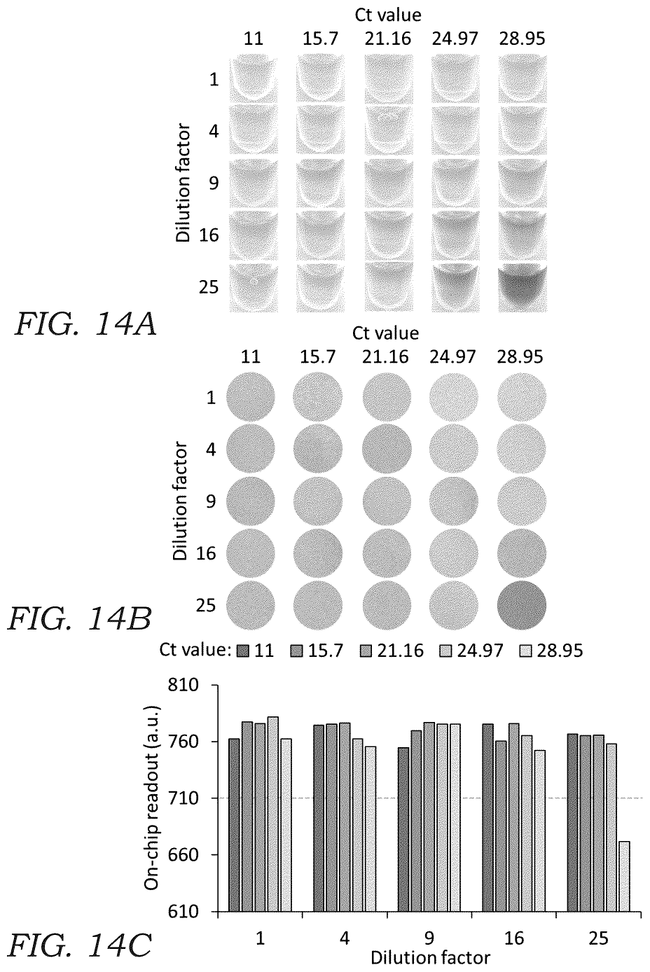

Prior to applying the scaled platform for pooled testing of clinical samples, the dilutive effect of sample pooling was evaluated on the assay detection capability (using positive nasal swab samples). The results indicated the assay capability in correctly identifying positive samples with a relatively low viral load, even at dilutions as high as 16 times (FIGS. 14A-14C).

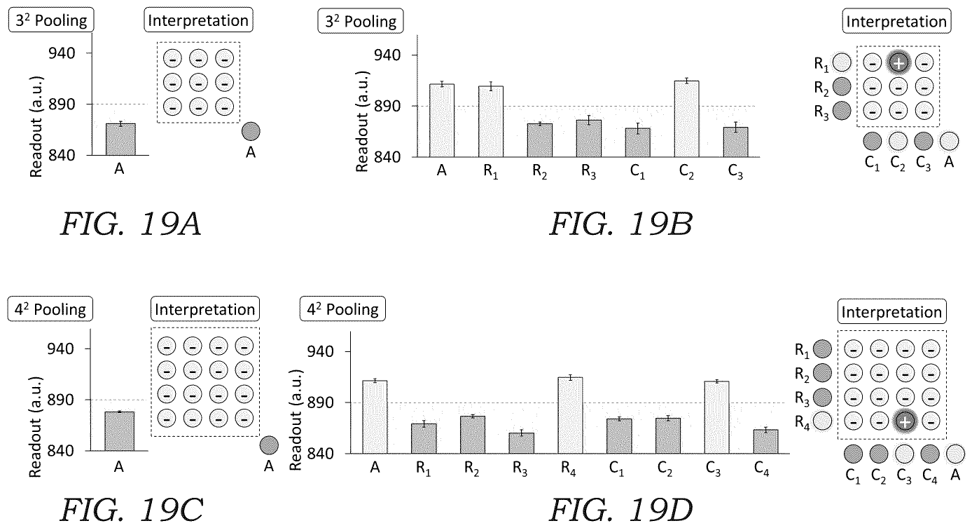

The pooled testing capability of the scaled platform was examined by analyzing a collection of fifty clinical samples (pre-characterized via RT-PCR). These samples were grouped in two arrangements of 9 and 16 samples and tested with the corresponding 3² and 4² microfluidic chips in a way to allow for evaluating the platform's pooling, detection, and interpretation capabilities in the first and second round of testing. Specifically, for each group size/chip, scenarios were tested that involved the absence or the presence of an infected sample. FIGS. 4C-4F illustrate the corresponding on-chip optical characterization results (with assay reagent volumes of 19 μL to analyze aliquoted samples with volumes of 1 μL). Following the aforementioned testing scheme, by comparing the corresponding assay responses (all-pooled, A or row/column-pooled, Rᵢ/Cⱼ) with respect to their detection threshold, the status of each sample was determined. Similar pooled testing studies were performed using smaller aliquoted samples (100 nL, with assay reagent volumes of 1.9 μL), demonstrating the ability to reduce reagents further (FIGS. 19A-19D). For all tested scenarios and across all samples, the ferrobotically produced/interpreted results were in line with those obtained by RT-PCR.

The demonstrated pooled-testing application, and scale of microfluidic liquid handling operations, is unprecedented. Table 1 provides a detailed account of the number of droplet actuation and ferrobotic operations that were reliably carried out to achieve pooled testing.

TABLE 1
Table 1: Breakdown of the ferrobotic operations
and commuted pixels for 4² pooled testing.

| | Rounds | | | | |
|---|---|---|---|---|---|
| | Round 1 | | | Round 2 | |
| | Sub rounds | | | | |
| | | 16 to 4 | 4 to 1 | Row | Column |
| | Aliquoting | Pooling | Pooling | Pooling | Pooling |
| Aliquoting | 32 | 4 | 1 | 4 | 4 |
| Disposing | 16 | 4 | 1 | 4 | 4 |
| Merging | 0 | 4 | 2 | 8 | 8 |
| Mixing | 0 | 4 | 1 | 4 | 4 |
| Collecting | 16 | 16 | 5 | 20 | 20 |
| Transportation | 48 | 28 | 8 | 32 | 32 |
| Total operations | 112 | 60 | 18 | 72 | 72 |
| | | 190 | 334 | 144 | |
| Commuted pixels | 276 | 932 | 245 | 904 | 908 |
| Total commuted | | 1453 | 3265 | 1812 | |
| pixels | | | | | |

This was achieved by harnessing the competitive advantages of the ferrobotic technology that overcomes performance limits (in terms of reliability, scalability, reagent use, portability, etc.) and cost barriers of alternative microfluidics approaches.

Depending on the situational needs, the ferrobotic testing device 10 can be adapted—with minimal reconfiguration—to automate other NAAT-based assays (e.g., RT-PCR) as well as other pooling schemes (e.g., Dorfman). The ferrobotic testing platform can be constructed with low-cost consumables (Table 2) and instrumentation (Table 3) using widely available materials and circuit components and following existing scalable manufacturing solutions; altogether enabling mass-production for rapid large-scale deployment.

TABLE 2
Table 2: Comparison of the cost of key materials of
a low-cost EWOD chip vs. a generic ferrobotic chip.

| Chip | Component | Cost per cm² (USD) |
|---|---|---|
| EWOD^{[7]} | PVA | 0.004 |
| | Parylene-C | 0.002 |
| | Teflon | 0.467 |
| | ITO glass | 0.240 |
| | Total | 0.71 |
| Ferrobotics | Ferrofluid | 0.001 |
| | Double-sided tape | 0.005 |
| | PET | 0.003 |
| | Novec 7500 | 0.0002 |
| | Pico-Surf | 0.0004 |
| | Total | 0.01 |

Abbreviations: polyvinyl alcohol (PVA), indium tin oxide (ITO), and polyethylene terephthalate (PET).

TABLE 3
Table 3: The list and quantity of constituent hardware components and
estimated cost of a ferrobotic platform with N testing channels.

| Component | Quantity | Cost (USD) |
|---|---|---|
| PCB board | [0.01N] | 25 × [0.01N] |
| Arduino Nano | [0.01N] | 5 × [0.01N] |
| LT3092 | N | 2.5N |
| MAX14662 | 2N | 5N |
| Resistors | 8N | 0.1N |
| BH1721 | N | 1.5N |
| LED | N | 0.1N |
| Total | | 30 × [0.01N] + 9.2N |
| Platform | 32 channels | 324.4 |
| | 96 channels | 913.2 |

[ ] denotes the ceiling function.
As summarized in Table 4, it was estimated that translating this platform for population-level screening can ultimately lead to ~3 orders of magnitude of increase in marginal gain in testing capacity from the instrumentation investment standpoint, and a 60 to 300-fold reduction in reagent costs at moderate-to-low viral prevalence (~8 to 0.8%) and 10-fold reduction at high viral prevalence.

TABLE 4
Comparison of the ferrobotic platform (N channels) and commercial
NAAT-based testing platforms. ┌ ┐ denotes the ceiling function.

| | | | | | Marginal | | | |
|---|---|---|---|---|---|---|---|---|
| | | | | | gain of | | | Area |
| | | | | | daily test | | | (cm²) |
| | | Reagent | Regent | | capacity | | Total | per |
| | | cost per | cost per | Turnaround | (per 1 M | Pooling | Area | testing |
| Platform | Cost | Assay | sample | Time (h) | USD) | capability | (cm²) | channel |
| Cobas Liat | $20,000 | $50 | $50 | 0.33 | 3.6 k | No | 270 | 270 |
| Analyzer | | | | | | | | |
| Liaison | $35,000 | $35 | $35 | 1.5 | 3.7 k | No | 650 | 81 |
| MDX | | | | | | | | |
| Cobas 6800 | $300,000 | $20 | $20 | 2.67 | 2.9 k | No | 63100 | 657 |
| KingFisher | $85,000 | $35 | $35 | 8 | 3.4 k | No | 5600 | 58 |
| Flex and | | | | | | | | |
| Applied | | | | | | | | |
| Biosystems | | | | | | | | |
| 7500 | | | | | | | | |
| Ferrobotic | 30 x | $.60 | $.6 | 0.67 | 3.8 M | Yes | 510 | 5.3 |
| Platform | [0.01 N] + | | p = 20% | | | | N = 96 | |
| | 9.2 N | | $.31 | | | | | |
| | | | p = 8.0% | | | | | |
| | | | $.07 | | | | | |
| | | | p = 0.8% | | | | | |

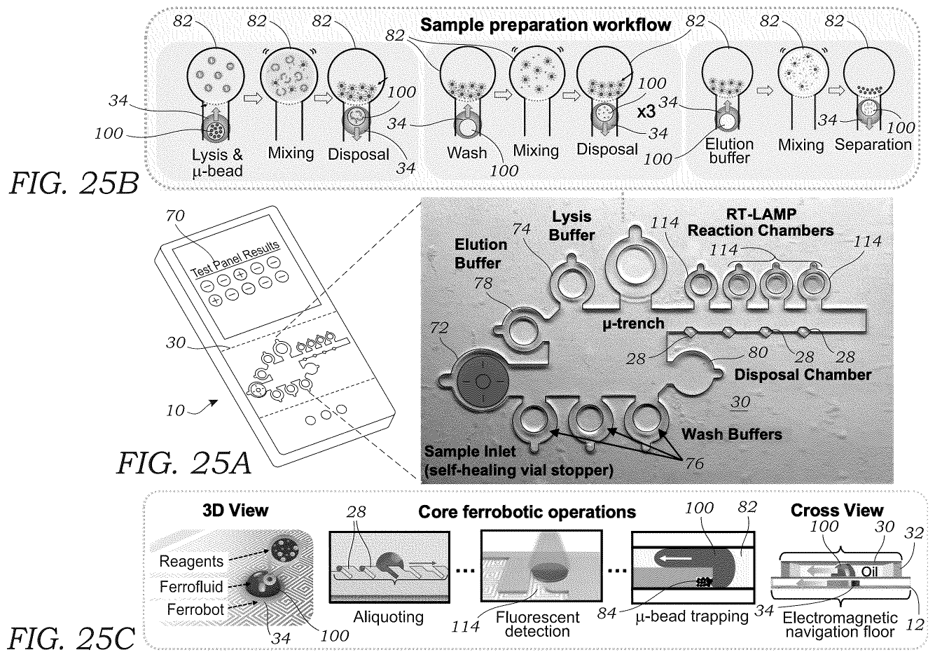

FIG. 25A illustrates another embodiment of a digital ferrofluidic fluid assay device 10. This device 10 is a hand-held viral diagnostic platform (approximately the size of a Smartphone) that allows for seamless, low-cost testing against a panel of pathogens (e.g., filoviruses) in extremely remote settings. As in the prior embodiments, the device 10 includes first substrate 12 (i.e., PCB) (located inside device 10) that contains the addressable coils 14 that are used to manipulate the one or more permanent magnets 34. A second substrate 30 in the form of disposable microfluidic chip or cartridge is disposed adjacent to the first substrate 12 as described previously and one or more permanent magnets 34 or ferrobots are controlled to manipulate volumes or droplets of ferrofluid 100 to perform one or more operations withing the microfluidic chip 30. In the embodiment of FIG. 25A, a single permanent magnet 34 can be used to perform all of the device operations although it should be appreciated that a plurality of such permanent magnets 34 could also be employed. The device 10 is battery-operated and miniaturizes, integrates, and automates magnetic bead-based sample preparation and multiplexed nucleic acid amplification workflows that are currently performed manually in standard lab settings. The device's key attributes in terms of automation, multiplexing of target viral nucleic acids or patients, portability, battery-operability, real-time connectivity, low-cost (instrument+cartridge), makes it a promising field-deployable viral diagnostic solution for decentralized settings.

As seen in FIG. 25A, the device 10 includes an optional display 70 that can be used to show test results to the user. The device 10 uses bead-based sample preparation and multiplexed nucleic acid amplification workflows to enable seamless low-cost testing against a panel of pathogens. The beads are magnetic beads that are functionalized to a target or target class of molecules or species. The target species may include nucleic acids, proteins, biomolecules, and the like. It is important to note that these magnetic beads differ from the magnetic particles 102 that are nanometer-scale particles within the droplets 100. These magnetic beads are larger in size such that a larger magnetic force can be applied to concentrate the beads to a smaller volume or location in the droplet 100. This enables the beads to enrich and concentrate target molecules from a sample. For example, as described herein, the beads are functionalized to bind to nucleic acids. The workflows are based on those currently performed manually following standard viral diagnostic practices and using commercially available reagents. However, with this device 10, bead-based sample preparation enhances the limit of detection (LoD) and allows for reliable analysis in multiple complex biomatrices including blood, saliva, and oral swab in viral transport media. FIG. 25A illustrate the second substrate or microfluidic chip 30 that includes a sample inlet 72 which may include a self-healing septum (e.g., vial stopper). A chamber 74 for storage of lysis buffer is provided along with wash buffer chambers 76 and an elution buffer chamber 78. A disposal buffer chamber 80 is provided for waste fluid storage. One or more assay chamber(s) 114 are located in the second substrate or microfluidic chip 30 and contain assay reagents therein. In one embodiment, the one or more assay chambers 114 contain RT-LAMP reagents (e.g., primers) and/or buffers. Different assay chambers 114 may contain different RT-LAMP reagents and/or buffers specific to different targets. This allows for multiplex analysis with a single microfluidic chip 30. For example, in one implementation, the different assay chambers 114 may contain test reagents or buffers specific to different viruses (e.g., Ebola Zaire (Filovirus), Ebola Sudan (Filovirus), Marburg (Filovirus), and Lassa Fever Virus (Arenavirus). The nucleic acid amplification workflow is based on the established LAMP methods allowing for simplifying the assay workflow and hardware requirements while delivering PCR-level performance. If needed PCR or other nucleic acid amplification workflows can also be miniaturized, integrated, and automated within the device 10. In addition to assays that identify the presence or absence of the target species in a sample, the assays may also be used to quantify the abundance of the target species in the sample.

The microfluidic chip 30 includes corrugation features 28 that are used for generating smaller sized volumes or droplets of ferrofluid 100 and/or sample or other reagents. In this embodiment, a trench chamber 82 is provided that is used to trap the magnetic beads that have affinity to target biomolecules in the sample, such as nucleic acids. In some embodiments, a plurality of such trench chambers 82 may be located in a single second substrate or microfluidic chip 30. The trench chamber 82 includes a trench recess 84 that aids in trapping the beads in the trench chamber 82. Because the volume or droplets of ferrofluid 100 contain magnetic beads therein, the magnetic beads are attracted to the magnetic field of the underlying permanent magnet 34 and are accumulated at the bottom surface and are retained in the trench recess 84 of the trench chamber 82 as the ferrofluid is pulled out of the trench chamber 82. In a related embodiment, other non-magnetic beads which are configured to bind biomolecule targets are introduced in the droplet 100. These non-magnetic beads comprise beads with a diameter that is larger than a gap size in the trench chamber 82, leading to accumulation and concentration of the non-magnetic beads, based on their larger size upon the movement of the ferrofluid droplet 100 through the trench chamber 82. With reference to FIG. 25B, the magnetic (and non-magnetic) approach allows various different fluids (e.g., lysis reagents, wash, elution buffers) to be mixed in the trench chamber 82 and then removed while leaving behind the magnetic beads. As explained, this enables the purification and enrichment of genetic material via elution into a much smaller volume of elution buffer. FIG. 25C illustrates a 3D view of a volume or droplet of ferrofluid 100 that contains the ferrofluid and reagents therein. Aliquoting is performed using the corrugated features 28. Fluorescent or colorimetric detection takes place in the assay chambers 114 using optical sensor(s) 48 as described herein. The magnetic beads are trapped in the trench chamber 82 as seen in FIG. 25B when the volumes or droplets of ferrofluid 100 are withdrawn from the trench chamber 82.

The entire testing process is operated within a disposable microfluidic chip 30, wherein the bioanalytical operations are controlled via a palm-size, battery-operated, programmable printed circuit board (PCB) 12 of the type disclosed herein located within the device 10. The microfluidic chip 30, which is millimeter-scale and easily mass producible through injection molding, contains reagent-carrying and sample-carrying ferrofluid volumes or droplets 100 (with surrounding oil) as well as a dedicated inlet/reservoir 72 for sample loading. For ease of sample loading, the 72 inlet is made with a self-healing rubber diaphragm, which is compatible with existing capillary/venous blood collector solutions (e.g., Tasso). The microfluidic chip 30 hosts all the core ferrofluid operations, including transportation, mixing, merging, splitting, and bead-based manipulation. Ferrofluid allows for magnetization of sample and magnetic actuation, and it is based on biocompatible magnetic nanoparticles (non-interfering with biological enzymes or other reactions). The device 10 is smartphone-sized (~7×15 cm²) and battery-operated (LiPo; used in smartphones). As explained, the PCB 12 includes a 2D array of coils 14 which can be independently activated (0.2 A) to electromagnetically direct millimeter-sized permanent magnets 34 or ferrobots, allowing for contactless manipulating ferro-droplets 100 within the overlying microfluidic chip 30. The PCB 12 also incorporates the required electronics (e.g., heater 42, temperature sensor, and optical detection hardware such as the optical sensing module 44) for colorimetric LAMP analysis. An on-board MCU 18, which is installed with the instruction software, allows for autonomously guiding the microfluidic operations involved in the multiplexed testing process.

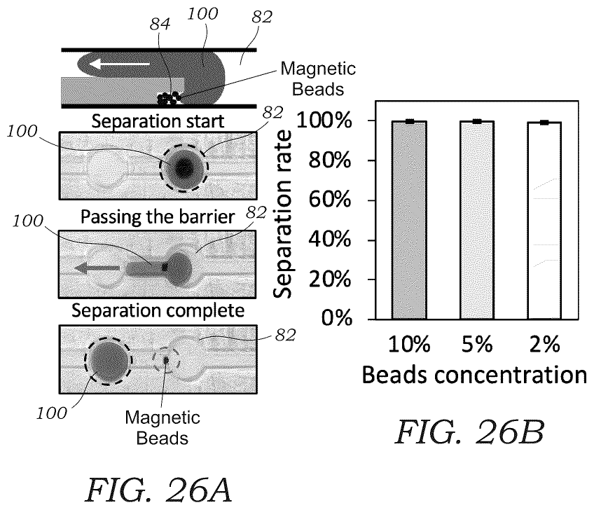

Sample preparation is one of the major technological barriers for decentralized viral diagnostics. The current commercial solutions are bulky and require extensive intermediary manual operations prior to performing nucleic acid amplification/detection steps (in separate instruments). The device 10 of FIGS. 25A-25C realizes on-chip sample preparation using a ferrobotic, magnetic bead-based nucleic acid extraction workflow. Isolation, purification, and concentration of viral nucleic acids is performed in this step to enable use of larger sample volumes to increase sensitivity, but smaller reaction volumes to save reagent costs and enable multiplexing for testing. In this workflow, and with reference to FIG. 25A, the permanent magnet 34 or ferrobot captures (isolates) the nucleic acid content in the ferro-droplets 100 by mixing them with ferro-droplets 100 containing lysis buffer solutions and magnetic bead-based nucleic acid capture agents (e.g., Applied Biosystems MagMAX CORE kit, available from e.g., Thermofisher). Next, by trapping the magnetic beads against a trench recess 84 in the trench chamber 82 (height~100 μm) and performing wash steps, the ferrobots 34 purifies the genetic material and then concentrates it, via elution into a 20-fold smaller volume of elution buffer. Elution buffer acts to release the biomolecules, such as nucleic acids, bound to the magnetic beads. Other means to release the biomolecules from the beads may be used including temperature modification, solvent addition, adjusting salt concentration, or adding other additives to achieve this effect. In this approach, the trench recess 84 in the trench chamber 82 also acts as a weir to separate the magnetic beads from the concentrated sample. FIG. 26A illustrates images of how the magnetic beads are separated using the trench chamber 82. FIG. 26B illustrates a graph showing the separation efficiency for different bead concentrations.

The permanent magnet(s) 34 or ferrobot(s) deliver the enriched samples to designated assay chambers 114 for multiplexed LAMP-based nucleic acid amplification. This on-chip sample preparation solution eliminates interfering abundant species prior to RT-LAMP, enabling reliable detection of a wide panel of pathogens in different complex biomatrixes (e.g., blood, saliva, oral swab-in-VTM). Furthermore, this solution improves the limit of detection by more than an order of magnitude, accomplished through the enrichment of nucleic acids from the larger sample volume into a smaller reaction volume. This is especially crucial for the detection of filoviruses, where RNA concentration is extremely low, and the extraction process must ensure the capture of few nucleic acid materials in a relatively large input volume.

Multiplexed viral testing is crucial for filovirus diagnosis and treatment due to the variation in disease severity caused by different types, despite similar initial clinical symptoms. The device 10 of FIGS. 25A-25C can perform multiple parallel RT-LAMP tests with a single sample input, and with the same level of sensitivity/specificity offered by singleplex RT-PCR. The microfluidic chip 30 includes corrugated features 28 for droplet aliquoting and assay chambers 114 that are pre-filled with RT-LAMP buffers (e.g., from standard kits) specific to a given target. The MCU 18 can be programmed to: 1) create multiple aliquots of an initial droplet sample; 2) deliver each aliquot to the corresponding assay chamber 114; 3) activate underlying resistive heaters 42 beneath each reaction buffer for target nucleic acid amplification (by maintaining constant temperature of 65 °C for 30 min); 4) optically quantify the test results with the aid of an on-board light source 46 and optical sensors 48 (e.g., array of single-wavelength optical sensors such as 560 nm-BH1721, ROHM Semiconductor; situated beneath each assay chamber 114); and 5) display the test results on an on-board digital screen or display 70.

The magnetic beads used in the device 10 may include commercially available functionalized or generic magnetic beads with optimized surface chemistry and sizes. To improve the limit of detection/response time, instead of a colorimetric response, the device 10 may include an optical sensing module 44 that operates on fluorescence-based detection. In this regard, optical interrogation of the assay chamber(s) 114 may be done by looking for a fluorescence signal or output from the assay chamber(s) 114. Alternatively, optical interrogation may come from a colorimetric change in the assay chamber(s) 114 as explained herein. It should be appreciated that the device 10 is adaptable to perform PCR, other nucleic acid amplification assays, as well as other assay workflows (involving bead-based or generally surface-phase/liquid-phase molecular reactions) beyond viral diagnostics.

Accordingly, leveraging its high level of accessibility, adaptability, and automation, the device 10 can be deployed as a democratized, distributed, and decentralized solution to expand testing capacity for pandemic preparedness. Beyond viral testing, the swarm ferrobotic technology can be adapted and scaled to efficiently streamline and massively parallelize a variety of other lab-based bioanalytical operations within a miniaturized footprint. Thus, this technology can serve as a powerful tool for a wide range of biomedical and biotechnological applications such as diagnostics, omics, drug development, and chemical/biomaterial synthesis.

### Materials and Methods

### Materials and Reagents for Ferrobotic Platform and Viral Testing

The ferrofluid used for the volumes or droplets of ferrofluid 100 was ferumoxytol, a U.S. Food and Drug Administration-approved intravenous iron preparation (AMAG Pharmaceuticals, MA, USA). Rare earth permanent magnets 34 (D101 and DH2H2, with corresponding thickness/diameter of 0.8 mm/2.54 mm and 5 mm/5 mm) were purchased from K&J Magnetics (PA, USA) for device 10 construction and characterization. The microfluidic module or second substrate 30 is constructed from double-sided tape (3M, 300LSE, MN, USA) and transparent PET film layers (M.G. Chemicals, Ontario, Canada). The microfluidic devices were filled with Novec 7500 Engineered oil (3M, MN, USA) containing 0% to 0.1% surfactant (Pico-Surf 1, Sphere Fluidics, NJ, USA) as the filler fluid 106, unless stated otherwise. Other oils including mineral oil (Sigma-Aldrich, MO, USA), FC-40 and corresponding surfactant (1H,1H,2H,2H-Perfluoro-1-decanol, Sigma-Aldrich, MO, USA) were also used for velocity characterization. Single stranded RNA (ssRNA) fragments of SARS-COV-2 (10⁸ copies/μL) were purchased from Sigma-Aldrich. Living E. coli K-12 strain (3×10⁵ CFU/μL) in liquid nutrient broth was purchased from Carolina Biological Supply (NC, USA). A SARS-COV-2 Rapid Colorimetric RT-LAMP Assay Kit was purchased from New England Biolabs (NEB, MA, USA) and stored at −20 °C The Viral Transport Media (VTM) was purchased from BD (NJ, USA). The UCLA Clinical Microbiology Laboratory performed RT-PCR using the following assay: TaqMan COVID-19 RT-PCR Assay (ThermoFisher Scientific, Carlsbad, CA, USA).

### Electromagnetic (EM) Navigation Floor Circuit Design

To manipulate the permanent magnets 34 or ferrobots across 2D space, an EM navigation floor on PCB 12 comprised active coil elements 14 in a 2D-array format. Each coil element 14 had a three-turn coil with a size of 1.5 mm by 1.5 mm traced onto the three layers of the PCB 12, with a 0.1 mm gap separated from adjacent coil elements 14. Each coil element 14 can be activated by a 0.2 A direct current (DC), generating a localized magnetic force that attracts the permanent magnet 34 or ferrobot. Programmable current source ICs LT3092 (Linear Technology, CA, USA) were used to power the actuated coils 14 (3 V, 0.6 W for each actuated coil 14). Programmable switch ICs MAX14662 (Maxim Integrated, CA, USA) 16A. 16B were used to selectively activate the EM coils 14 and components.

To enable scalable asynchronous parallel testing of 32 samples, the individual testing navigation floor comprised an array of 4×8 testing units. Each testing unit included two 20 Ohm resistive heaters 42, an array of fourteen (14) EM coils 14, and an optical sensing module 44 containing a white-light LED as the light source 46 (20 mA) and an ambient light sensor 48 (3.3 V) with 560 nm peak absorbance (BH1721, ROHM Semiconductor, Japan). The optical components are operated with stable supply conditions, minimizing signal drift.

Each EM coil element 14 was individually addressed by the output of a switch IC 16A, 16B. The matrix-format navigation floor was designed for general ferrobotic operations and testing applications, comprising an active matrix array of EM coil elements 14, which was specifically selected when switch ICs 16A, 16B activate corresponding rows and columns in the navigation floor (FIG. 7F). Switch ICs 16A, 16B were controlled via Serial Peripheral Interface (SPI) by an Arduino Nano 18, which in turn communicated with a computer 20 through serial communication. Target coordinates preprogrammed or sent from the user interface were translated to SPI commands by the Arduino 18 and then transmitted to switch ICs 16A, 16B for addressable activation of the EM coils 14. The EM navigation floor 12 can be functionalized with benchtop instruments (laptop and power supplies) or as a self-sufficient battery-operable handheld unit (FIGS. 23A-23B).

### Microfluidic Device Fabrication

The microfluidic chips or second substrate 30 were fabricated by assembling layers of double-sided tape and transparent polyethylene terephthalate (PET) film sheets. In particular, one and six layers of double-sided tape were used to construct microfluidic chips with corresponding heights of ~150 μm and 900 μm. Patterns were laser cut into the double sided-tape and PET to make micro-channels (VLS 2.30, Universal Laser System, AZ, USA). The double-sided tapes and PET sheets were then thoroughly cleaned by immersing them in an acetone ultrasonic bath for 5 min, followed by repeating this cleaning process with isopropyl alcohol and deionized water. To completely dry the cleaned microfluidic layers, the devices were baked at 65 °C for 4 h. In order to make the surface of the microchannels hydrophobic, the inner surface of double-sided tape and PET sheets were exposed by a shadow mask and treated with NeverWet base-coat spray (Rust-Oleum, IL, USA), followed by resting for 30 min. The devices were then again treated with NeverWet top-coat spray, followed by room temperature incubation for 12 h. The droplet merging electrodes 36 were patterned on PET sheets by photolithography using positive photoresist (AZ5214E, MicroChemicals, Germany), followed by the evaporation of 20 nm of Cr and 100 nm of Au and a lifting-off step in acetone. The fabricated microfluidic devices 30 were preloaded with oils containing various concentrations of surfactants for the filler fluid 106 and reagents for experiments.

Maximum Transportation Velocity Characterization within Different Oil Environments

Microfluidic devices 30 with 50-mm by 30-mm by 0.7-mm inner chambers were fabricated and assembled. A ferrobot 34 (or permanent magnet) was placed on top of the navigation floor 12 and below the microfluidic device 30 (in the gap G area). Microfluidic chambers filled with different oils including mineral oil, FC-40 (w/or w/o 5% Perfluoro) or Novec-7500 (w/or w/o 0.01% Pico-Surf) were used for velocity characterization. After the ferrofluid droplets 100 (2 μL) were loaded in the microfluidic chambers, these droplets 100 moved along with the ferrobot 34, which was sequentially guided by the EM coils 14 actuation in an array from left to right. The velocity of the ferrobot 34 was controlled by adjusting the time interval between activating two adjacent coils 14. If the ferrofluid droplet 100 followed the ferrobot 34 to the end successfully, then the velocity of the magnet 34 would increase by shortening the actuation time interval (by 1 ms) in the next round until the droplet 100 failed to follow the magnet 34.

### Droplet Aliquoting Characterization Setup and Procedure

To validate the aliquoting operation in the optimized oil environment, microfluidic devices 30 (with heights of ~150 μm or 900 μm) containing various corrugated wall features 28 were designed. Devices 10 with different opening widths (0.2, 0.4, 0.8, 1.2, and 1.6 mm) at the corrugated wall were fabricated, assembled, and tested. After a parent ferrofluid droplet 100 was loaded in each device 10 (2 μL and 10 μL for devices with channel heights of 150 μm and 900 μm, respectively), it was transported by the ferrobot 34 along the same-sized repeated corrugated structures 28 to aliquot smaller ferro-droplets 100. The aliquoted droplets 100 were imaged to measure the droplet size.

### Merging Characterization Setup and Procedure

A microfluidic device 30 for merging and mixing was fabricated and assembled, with patterned electrocoalescence electrodes 36 (1 mm width, spaced 2 mm apart, thicknesses of 20 nm of Cr and 100 nm of Au) on PET substrate. To characterize merging, after two 5 μL ferrofluid droplets 100 were loaded in each microfluidic device 30 filled by Novec 7500 with different Pico-Surf surfactant concentrations (0.01%, 0.05%, 0.1%, 0.5%, 1%) as the filler fluid 106, the two droplets 100 were manipulated by the ferrobot 34 to the vicinity of the actuation electrode 36. A gradually increased (0.1 V increments) DC voltage was applied between the two electrodes 36 until the droplets 100 merged.

### Mixing Characterization Setup and Procedure

To characterize active mixing, the device 10 was loaded with one 5 μL colored ferrofluid droplet 100 and one 5 μL transparent water droplet. After merging, the underlying ferrobot 34 was directed to induce chaotic fluid motion within the merged droplet 100 with different frequencies (0.2, 1, 3, 5 Hz). A video recording was taken for the mixing process, and the droplet homogenization rate was calculated through image processing. To quantify mixing efficiency, the video frames were imported into a MATLAB, and the pixel data (in grayscale) at the droplet region were extracted. A mixing index is defined, as expressed below:

$$Missing index = 1 - \sqrt{\frac{1}{N - 1} {\sum }_{i = 1}^{N} \frac{{( {c}_{i} - {c}_{ave} )}^{2}}{{c}_{ave}^{2}}}$$

where N, cᵢ, and cₐᵥₑ are the total number of pixels, the grayscale values at pixel i, and the average grayscale values over N pixels, respectively.

### Characterization of Long-Term Cyclic Ferrobotic Operations

A microfluidic device 30 that contains two chambers and a connection channel in between was fabricated and assembled. The connection channel contains a corrugated wall feature 28 and a pair of merging electrodes 36 deposited on the PET substrate. After a 7.0-μL ferro-droplet 100 was loaded into the microfluidic chamber, the ferrobot 34 manipulated the ferro-droplet 100 periodically: dispense the droplet 100 into mother and daughter droplets 100 when transporting from the right chamber to the left chamber, and merge the mother droplet 100 with the dispensed droplet 100 when transporting from the left chamber to right chamber. These actions were repeatedly performed for more than 800 cycles. Images were taken during the whole process, and the dynamic variation of the droplet size was measured through image analysis. To illustrate the extreme reliability of the ferrobotic droplet actuation across different ionic strength and chemical conditions, 10 droplets with differing compositions (H2O, PBS, 0.1 M and 1 M HCl, 0.1 M and 1 M KCl, 0.1 M and 1 M NaCl, 0.1 M and 1 M NaOH) were actuated by designated ferrobots 34 over more than 70,000 cycles (12 commuted pixels-per-cycle/ferrobot) and 24 h. The actuation events and commuted pixels are tracked by monitoring the current through the designated impedance sensing gold electrode pairs (with the aid of CH Instrument 660E, TX, USA; applied voltage: 1 V).

### Programmable Heating Characterization Setup and Procedure

To implement programmable heating, a microfluidic device 30 was placed on the PCB 12 that contained resistive heaters 42. The heated region in the microfluidic chip 30 was placed right above the location of the resistive heater 42. Copper cubic blocks (3 mm length, 0.8 mm width, 2 mm height) were placed between the surface of the PCB 12 and microfluidic chip 30 for heat transduction. To characterize the heating function, different currents were applied through the resistive heater 42 (0-0.14 A), inducing a temperature increase by Joule heating. The temperature was then measured by a thermocouple. By programming the current through the resistive heater 42, the local temperature can be set in relation to the surrounding temperature (FIGS. 16B, 16C). If increased precision control of local temperature is desired, a temperature sensor can be integrated to form an internal real-time closed-loop temperature control mechanism.

### Off-Chip RT-LAMP Characterization

To detect RNA, RT-LAMP assays were conducted at room temperature. As the standard protocol described by NEB, every 25 μL RT-LAMP assay included 12.5 μL WarmStart Colorimetric RT-LAMP 2× master mix, 2.5 μL guanidine hydrochloride, 2.5 μL target RNA primer mix, 5.5 μL nuclease-free water, and 2 μL input sample. To characterize the RT-LAMP assay for SARS-COV-2 detection, ssRNA fragments of SARS-COV-2 diluted to various concentrations (0, 25, 100, 1000 copies/μL) were mixed with the assay as input sample, then the RT-LAMP assays were incubated at 65 °C for 30 minutes. After incubation, the assays were further analyzed by Nanodrop One (Thermo Fisher Scientific, MA, USA) and gel electrophoresis. Plate reader Cytation 5 (BioTek, VT, USA) was also used to record the assay absorbance (at 560 nm) during incubation (at 65 °C) using a 384 well plate.

RT-LAMP Characterization within the Ferrobotic Chip

To characterize the RT-LAMP assay performance within the ferrobotic microfluidic chip 30, a 20 μL-RT-LAMP assay containing ferrofluid volumes or droplets 100 was prepared for on-chip reaction. The compositions of the assay are: 10 μL WarmStart Colorimetric RT-LAMP 2× master mix, 2 μL guanidine hydrochloride, 2 μL target RNA primer mix, 5 μL nuclease-free water and 1 μL input sample. The input sample contained ssRNA fragments of SARS-COV-2 in various concentrations (0, 25, 100, 1000 copies/μL) and 13% of ferumoxytol. The RT-LAMP assays were loaded in the assay chamber 114 and incubated at 65 °C for 30 minutes. The incubation process of the assay was recorded by video. After the RT-LAMP reaction, the color was quantitatively measured by the optical sensors 48. Similar procedure was performed with a 2 μL-RT-LAMP assay and using microfluidic devices 30 with reduced height (~150 μm), when characterizing the assay's response to 100 nL-input samples.

### Standard RT-PCR Test for Clinical Samples

The TaqPath COVID-19 RT-PCR assay targets the SARS-COV-2 S, N and ORF1ab genes. Extraction was performed on the automated KingFisher Flex Purification System. RT-PCR was performed on the Applied Biosystems 7500 Real-Time PCR Instrument. Detection of two or more targets was considered positive. All testing was performed on nasopharyngeal swabs collected from symptomatic patients. The CT values were extracted from each instrument and represent a midpoint between the target genes.

### Off-Chip RT-LAMP Detection for Clinical Samples

All clinical samples were obtained following University of California, Los Angeles, Institutional Review Board Approval (IRB #21-000982). Clinical samples were collected using a nasal swab, stored in the VTM at −80 °C, and added into PBS buffer (20% VTM+80% PBS buffer) with inactivation reagent (including 6 mM NaOH for adjusting pH, 2.5 mM TCEP-HCl, 1 mM EDTA). For the off-chip RT-LAMP test, the samples were placed in a heat block set to 95 °C for 5 min to be inactivated. RT-LAMP assays were prepared following the off-chip protocol and incubated at 65 °C for 30 minutes. Optical images were taken after the incubation.

### Ferrobotic Individual Clinical Sample Testing

To perform ferrobotic SARS-COV-2 individual tests on clinical samples, microfluidic devices 30 containing one or more sample holding chambers or regions 112 (e.g., input chamber), a ferrofluid holding chamber 110, an assay chamber 114, two pair of merging electrodes 36 (patterned at the sample holding chamber or region 112 and assay chamber 114) and a dispensing structure that included a corrugated feature 28 was fabricated and assembled. Each microfluidic chip 30 was preloaded with a ferrofluid droplet 100 (50% ferumoxytol) in the ferrofluid chamber 110 and RT-LAMP assay solution in the assay chamber 114 (reagent volume: 1.9 μL and 19 μL for analysis of 100-nL and 1 μL-aliquoted samples, respectively). For clinical sample analysis, the starting sample was pipetted into the microfluidic chip 30 at the sample holding chamber or region 112 (i.e., input chamber via the designated sample inlet). Specifically, 0.52 μL and 5.2 μL of starting samples were correspondingly used for subsequent aliquoting/analysis of 100-nL and 1 μL droplets. Then heat inactivation and lysis was performed on the PCB 12 for 5 minutes by powering a 20-Ohm resistive heater 42 with 0.14 A DC current. Thereafter, a ferrobot 34 performed the sample processing steps of transportation, merging, mixing, aliquoting, disposal, and delivery to the assay chamber 114 (FIGS. 11A-11B). Then, the on-chip RT-LAMP reaction (at 65 °C) continued for 30 min. The assay readout was measured by the optical sensing module 44.

### Ferrobotic Multiplexed Testing

For multiplexed detection of SARS-COV-2, H1N1, and rActin RNA, a microfluidic device 30 with a sample holding chamber or region 112 (i.e., input chamber), a ferrofluid chamber 110, an array of assay chambers 114, two pairs of merging electrodes 36 (patterned at the sample holding chamber or region 112 and across the array of assay chambers 114), and a dispenser array was fabricated and assembled. The dispenser array was formed by corrugated features 28 that function to create defined volumes or droplets 100 and/or sample. Each microfluidic chip 30 was preloaded with a ferrofluid droplet 100 in the ferrofluid chamber 110 and three 19-μL RT-LAMP reaction solutions, containing primers for SARS-COV-2, H1N1 (Thermo Fisher Scientific, MA, USA), and internal control (NEB, MA, USA) respectively, in the array of assay chambers 114. When performing a validation test, a blank sample or negative nasal swab sample either with or without target (spiked with SARS-CoV-2 and/or H1N1 positive control) was loaded into the microfluidic chip 30 at the sample holding chamber or region 112. Inactivation/lysis was then performed on the PCB 12 for 5 minutes by powering a 20-Ohm resistive heater 42 with 0.14 A DC current. Thereafter, a ferrobot 34 performed the sample processing steps of transportation, merging, mixing, aliquoting, disposal, and delivery to the assay chambers 114. Each RT-LAMP assay solution ended up receiving a 1 μL ferro-sample. Then, the on-chip RT-LAMP reaction (at 65 °C) continued for 30 min. The readout for each assay was measured by the optical sensing module 44.

### Ferrobotic Pooled Clinical Sample Testing

For pooled tests of clinical samples, microfluidic devices 30 with a matrix array of sample holding chamber or regions 112, dispensers (formed using corrugated features 28), two arrays of assay chambers 114 and five pairs of merging electrodes 36 (patterned across the array of assay chambers 114 and mixing regions) were fabricated and assembled. The assay chambers 114 were preloaded with RT-LAMP assay solutions (reagent volume: 1.9 μL and 19 μL for 100-nL and 1 μL-aliquoted samples, respectively). A number of 3.5-μL heat-inactivated starting ferro-samples were loaded into the input chambers (9 for 3², 16 for 4² pooling testing). Thereafter, ferrobots 34 performed the sample processing steps of several rounds of aliquoting, transportation, merging, mixing, and delivery to the corresponding assay chambers 114. The navigation planning of the ferrobots 34 accounted for the maintenance of an inter-ferrobot distance of 10 mm to avoid inter-ferrobot magnetic interference. The on-chip RT-LAMP reaction took place for 30 min (at 65 °C). The assay readout was measured by the optical sensing module 44.

### RT-LAMP Validation in Diluted Clinical Sample

Five nasal swab samples (originally obtained from COVID-19 infected donors, pre-characterized via RT-PCR) with various Ct values (11, 15.7, 21.16, 24.97, and 28.95) were diluted in PBS with different dilution rates (4, 9, 16, and 25). Then, all the diluted and undiluted samples were tested by both standard off-chip RT-LAMP and on-chip RT-LAMP testing. The reaction products of standard off-chip RT-LAMP were visually recorded in tubes. The reaction products of on-chip individual RT-LAMP were visually recorded in the microfluidic chips 30, then quantitatively measured by the optical sensing module 44.

### Mathematical Considerations for Individual and Square Matrix Pooled Testing

### Individual Testing

For individual testing, all the n samples (representing n patients) are tested once. Therefore, the total number of tests (Tₜₒₜₐₗ) for individual testing regardless of the viral prevalence (p) is:

Tₜₒₜₐₗ=n

Accordingly, as plotted in FIG. 1D, the number of tests per person using this approach is:

$$\frac{{T}_{total}}{n} = 1$$

### Square Matrix Pooled Testing

In this pooled testing approach, patient samples are grouped in formations of N=L² samples, arranged in a square matrix (L×L) format. Therefore, to test n patient samples via pooled testing, the number of formed groups (k) equals to:

$$k = \frac{n}{N} \qquad ( 1 )$$

Random Variables and their Corresponding Probabilities:

For a viral prevalence of p, one can assume the probability of a sample being positive is the same for each sample and equals to p, likewise the probability of one sample being negative equals to 1−p. This situation can be modeled as a binomial trial (or Bernoulli trial), wherein each sample has two possible outcomes: “positive” or “negative”. A random variable X is defined as the number of positive samples in the pooled group (with corresponding possible values x∈{0, 1, 2, . . . , N}). A random variable Y, was also defined which represents whether all the positive samples are in the same row/column or not (y∈{true, false}).

The probability of all samples in the pooled group of N samples being negative (i.e., the number of positive samples is equal to zero) can be expressed as:

P(X=0)=(1−p)^{N} (2)

Likewise, the probability of at least one sample being positive in the pooled group can be expressed as:

P(X>0)=1−(1−p)^{N} (3)

More generally, the probability of X being equal to a given value of x follows the binomial distribution:

$$P ( X - x ) = {{p}^{x} ( 1 - p )}^{N - x} ( \frac{N}{x} ) \qquad ( 4 )$$

The probability of random variable Y being ‘false’ can be derived based on the condition of X=x:

$$P ( Y = false \vert X = x ) = \{ \begin{matrix}0 & x \leq 1 \\ 1 - 2 {\prod }_{d = 1}^{x - 1} \frac{L - d}{{L}^{2} - d} & x > 1\end{matrix} \qquad ( 5 )$$

Based on the conditional probability relationship of P(A∩B)=P(B)P(B), the probability of X=x and Y=false can be expressed as:

$$P ( Y = false ⋂ X = x ) = P ( Y = false \vert X = x ) {{p}^{x} ( 1 - p )}^{N - x} ( \frac{N}{x} ) \qquad ( 6 )$$

Square matrix pooled testing may necessitate up to three rounds of sample pooling/testing.
First round: The samples in each group will be pooled as one sample, which is then tested by a dedicated assay (determining whether all samples are negative, or at least a positive sample is present). Since the first round is necessary for all groups, the number of tests in the first round (T₁) always equals to:

T₁=1 (7)
Second round: The prerequisite for this round is at least one positive sample exists in the pooled group of samples (X>0). In this round, samples are pooled along L rows and L columns, leading to 2L sample aggregates for testing. Accordingly, the number of tests in the second round (T₂) can be expressed as:

$${T}_{2} ( x ) = \{ \begin{matrix}0 , & x = 0 \\ 2 L , & x > 0\end{matrix} \qquad ( 8 )$$

Then, the expected number of tests in the second round (E[T₂(X)]) can be calculated as:

$$E [ {T}_{2} ( X ) ] = {\sum }_{all possible x} {T}_{2} ( x ) P ( X = x ) = {T}_{2} ( 0 ) P ( X = 0 ) + {T}_{2} ( x > 0 ) P ( X > 0 ) \qquad ( 9 )$$

According to (2), (3) and (8):

E[T₂(X)]=2L(1−(1−p)N) (10).
Third round: The prerequisite for this round is Y=false. During the third round, all the suspicious samples at the intersections of positive rows and columns are tested individually. Therefore, for a given x positive samples, in the worst case, x rows and x column will become positive, necessitating performing x² individual tests. It should also be noted that the number of tests cannot be greater than N. Therefore, the maximum number of tests in the third round

$$( {T}_{3}^{max} )$$

can be expressed as:

$${T}_{3}^{max} ( x , y ) = \{ \begin{matrix}0 , & y = true \\ min ( {x}^{2} , N ) , & y = false\end{matrix} \qquad ( 11 )$$

The probability of needing to perform the third round can be calculated as:

$${P}_{r o u n d 3} = {\sum }_{all possible x} P ( X = x ⋂ Y = false )$$

$${P}_{r o u n d 3} = {\sum }_{x = 2}^{N} P ( Y = false \vert X = x ) {{p}^{x} ( 1 - p )}^{N - x} ( \begin{matrix}N \\ x\end{matrix} )$$

For example, for the group size of N=4², P_{round3} will be ~2.5% at p=2% and ~0.7% at p=1%.

The expected number of tests in the third round (E[T₃(X,Y)]) can be approximated based on the derived expression for the maximum number of required tests in this round:

$$( 12 )$$

$$E [ {T}_{3} ( X , Y ) ] ∼ {\sum }_{all possible x and y} {T}_{3}^{max} ( x , y ) P ( X = x ⋂ Y = y ) = {\sum }_{x = 2}^{N} [ {T}_{3}^{max} ( x , true ) P ( X = x ⋂ Y = true ) + {T}_{3}^{max} ( x , false ) P ( X = x ⋂ Y = false ) ]$$

According to (5), (6) and (11):

$$E [ {T}_{3} ( X , Y ) ] ∼ {\sum }_{x = 2}^{N} min ( {x}^{2} , N ) P ( Y = false \vert X = x ) {{p}^{x} ( 1 - p )}^{N - x} ( \begin{matrix}N \\ x\end{matrix} )$$

### The Expected Total Number of Tests:

In the scope of testing the samples in each group, the expected total number of tests is the summation of the expected number of tests for each round of pooling:

$$( 14 )$$

$$E ( T ( X , Y ) ) = {T}_{1} + E ( {T}_{2} ( X ) ) + E ( {T}_{3} ( X , Y ) ) = 1 + 2 L [ 1 - {( 1 - p )}^{N} ] + {\sum }_{x = 2}^{N} min ( {x}^{2} , N ) P ( Y = false \vert X = x ) {{p}^{x} ( 1 - p )}^{N - x} ( \begin{matrix}N \\ x\end{matrix} )$$

Therefore, in the scope of testing all the n patients' samples (based on the k formed groups), the expected total number of tests (Tₜₒₜₐₗ) is given by:

$$E ( {T}_{t o t a l} ) = k \cdot E ( T ) = \frac{n}{N} E ( T ) \qquad ( 15 )$$

Accordingly, the expected required number of tests per person to find all infected samples can be expressed as (plotted in FIG. 1D for N=3² and N=4²):

$$( 16 )$$

$$\frac{E ( {T}_{t o t a l} )}{n} = \frac{1}{N} E ( T ) = \frac{1}{N} + \frac{2 L}{N} [ 1 - {( 1 - p )}^{N} ] + \frac{1}{N} {\sum }_{x = 2}^{N} min ( {x}^{2} , N ) P ( Y = false \vert X = x ) {{p}^{x} ( 1 - p )}^{N - x} ( \begin{matrix}N \\ x\end{matrix} )$$

Theoretical Analysis of Maximum Ferro-Droplet Transportation Velocity within Different Oil Environments

Here, a force-balance model was formulated to estimate maximum velocity as a function of system parameters. In the model, droplet kinematics are determined by three forces: a magnetic body force F_{M} acting on the ferrofluid droplet 100, a friction force between the droplet and channel surface F_{f}, and a drag force on the droplet in an oil environment F_{drag}. Droplet deformation by shear was ignored because of the small capillary number (an indicator of the relative strength of viscous forces in the presence of surface tension; here<0.01, Table 5).

The magnetic body force can be expressed as:

$${F}_{M} = \frac{{V}_{M} χ}{{μ}_{0}} ( B \cdot ∇ ) B \qquad ( 17 )$$

where V_{M} is the magnetically actuated volume of the ferrofluid droplet, μ₀ is permeability of free space, and B is the magnetic flux density. χ is the magnetic susceptibility (proportional to ferrofluid concentration), which can be equivalently expressed as χ=cχ₀ (c: volume ratio of ferrofluid; χ₀: magnetic susceptibility of 100% ferumoxytol).

The frictional force between the ferrofluid droplet and channel surface is on the order of:

F_{f}~K_{f}R_{b}μₒᵢₗU (18)

where K_{f} is the friction constant, R_{b} is the radius of the contact area between ferrofluid droplet and channel surface, μₒᵢₗ is the viscosity of the oil lubrication layer, and U is the velocity of the carrier (assuming that viscous drag in the vicinity of the contact line is significant).

Assuming that the droplet motion is in near-steady state, and given the relatively small value of the Reynolds number (<75, Table 5), drag force is on the order of:

$${F}_{drag} ∼ 3 π D {μ}_{o i l} U \frac{1 + 2 {μ}_{o i l} / 3 {μ}_{ff}}{1 + {μ}_{o i l} / {μ}_{ff}} \qquad ( 19 )$$

where D is the diameter of the droplet and u_{ff} is the viscosity of the ferrofluid.

During the process of magnetic actuation, the driving force F_{M} is counteracted by the restraining forces F_{f} and F_{drag}, establishing an upper-bound velocity (i.e., terminal velocity) for the droplet motion, which can be expressed as:

$$U \leq \frac{\frac{{V}_{M} {χ}_{0} c}{{μ}_{0}} ( B \cdot ∇ ) B}{{K}_{f} {R}_{b} {μ}_{o i l} + 3 π D {μ}_{o i l} \frac{1 + 2 {μ}_{o i l} / 3 {μ}_{ff}}{1 + {μ}_{o i l} / {μ}_{ff}}} = {U}_{max} \qquad ( 20 )$$

Rearranging the above equation leads to the scaling relationship between the maximum velocity and the viscosity of the surrounding oil.

$${U}_{max} = \frac{α c}{( β + \frac{1 + 2 {μ}_{o i l} / 3 {μ}_{ff}}{{μ}_{o i l} / {μ}_{ff}} ) {μ}_{o i l}} \qquad ( 21 )$$

$$where$$

$$α = \frac{\frac{{V}_{M} {χ}_{0}}{{μ}_{0}} ( B \cdot ∇ ) B}{3 π D} , β = \frac{{K}_{f} {R}_{b}}{3 π D} \qquad ( 22 )$$

According to (21), maximum velocity increases with increased ferrofluid concentration ratio c and decreased viscosity of surrounding μₒᵢₗ.

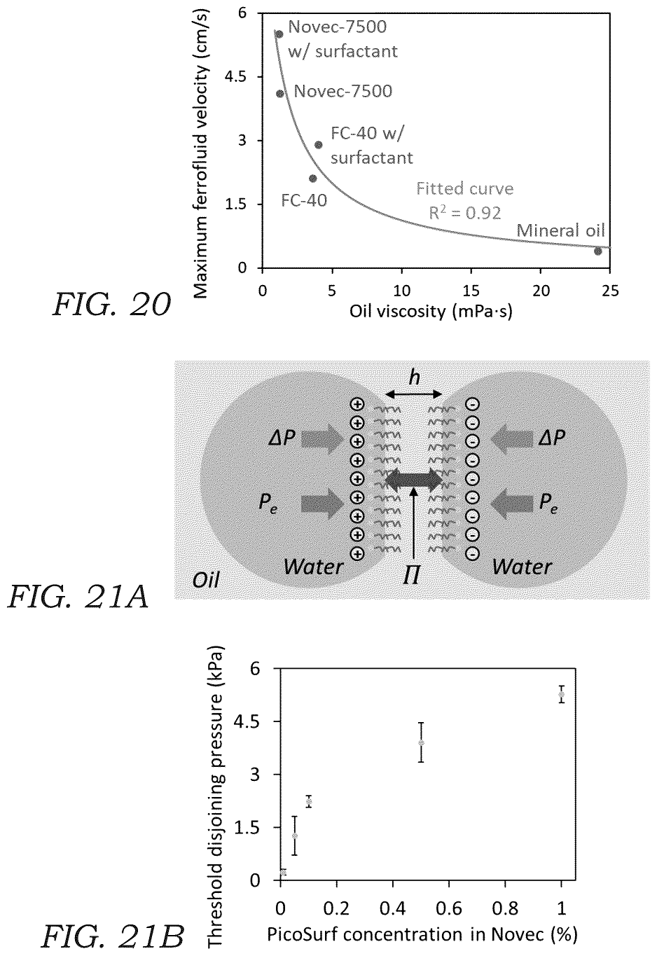

To estimate the parameters, u_{ff} (2 mPa·s) and μₒᵢₗ (Table 5) were measured by spinning disk rheometry. The parameters α and β from the scaling analysis were used to fit the experimental data (FIG. 20) resulting in values of α=177 (μN/m) and β=0.716 (R₂=0.92).

It is worth noting that introducing surfactant in the surrounding oil environment can increase the drag force, specifically, by modulating the boundary condition between the external phase and internal phase, creating interfacial tension gradients, and dampening the strength of internal flows in a droplet driven by the external flow. However, this effect appears to be small in the system, since increases in velocity for oil conditions containing surfactants was observed. Surfactants also improve shape deformation of the ferro-droplet by increasing the capillary number (Table 5), which helps the droplet follow the permanent magnet 34 during the transportation as it deforms. This effect could also contribute to the increase in velocity, because the droplet can sample higher magnetic field gradient regions and be actuated by higher magnetic body force.

### Theoretical Analysis of Threshold Voltage for Electrocoalescence at Different Surfactant Concentrations

Here, a force-balance model was used to explain the dependence of electrocoalescence threshold voltage on surfactant concentration. In the model, the interface between two water-phase droplets in the oil (containing surfactant) are determined by three pressures (FIG. 21A): an electric compression pressure Pₑ and a Laplace pressure ΔP that squeeze the water-oil interfaces and induce merging, as well as a repulsive disjoining pressure Π (originating from surfactant molecules aggregating at the interface, countering the direct contact of the droplets and merging).

The electric compression pressure at the water-oil interface can be expressed as:

$${P}_{e} = \frac{{ε}_{0} {ε}_{r} {V}^{2}}{2 {h}^{2}} \qquad ( 23 )$$

where ε₀ is vacuum permittivity, εᵣ is the relative permittivity of oil, V is the applied voltage, h is the thickness of the oil film.

The Laplace pressure at the water-oil interface can be expressed as:

$$Δ P = \frac{2 γ}{R} \qquad ( 24 )$$

where R is the radius of the droplet, γ is the surface tension of the interface.

The electrocoalescence of two droplets occurs when the combined electric compression pressure Pₑ and Laplace pressure ΔP exceed the upper bound of the disjoining pressure Π (i.e., threshold disjoining pressure, Πₜₕ):

Pₑ+ΔP≥Πₜₕ (25)

The effect of Laplace pressure can be neglected here, given that it is much smaller than the electric compression pressure in the experiment setting: Pₑ/ΔP=183 (assuming εᵣ=5.8, characteristic applied voltage V=1 V, characteristic oil film thickness h=100 nm, surface tension γ=7 mN/m, characteristic droplet radius R=1 mm).

As a result, the lower bound of applied voltage V (i.e., threshold voltage, Vₜₕ) to induce electrocoalescence can be estimated as:

$$V \geq \sqrt{\frac{2 {h}^{2} {Π}_{th}}{{ε}_{0} {ε}_{r}}} = {V}_{t h} \qquad ( 26 )$$

The threshold disjoining pressure Πₜₕ, and correspondingly Vₜₕ (∝\sqrt{Πₜₕ}), increase with the increasing concentration of the introduced surfactant. This relationship is in line with the trend observed in the experimental results shown in FIG. 2C. Furthermore, by rearranging (1) and referring to experimentally determined Vₜₕ values, the value of Πₜₕ at different surfactant concentrations can be derived (FIG. 21B):

$${Π}_{t h} = \frac{{ε}_{0} {ε}_{r} {V}_{th}^{2}}{2 {h}^{2}} \qquad ( 27 )$$

The derived trend shown in FIG. 21B is aligned with those obtained in previous studies.

### Ferrobotic Multiplexed Viral Testing

The ferrobotic platform's adaptability in delivering versatile fully automated assay workflows can also be exploited to perform multiplexed viral testing. This testing mode is diagnostically useful for differentiating between the emergent outbreak virus and the endemic viruses (e.g., the seasonal ones) that often present similar clinical symptoms. To illustrate this point, the automated platform was adapted to simultaneously test for the presence of SARS-CoV-2, Influenza H1N1, and rActin (an endogenous housekeeping gene, practically serving as an internal control). Accordingly, the disposable microfluidic chip 30 layout was revisted. Specifically, the microfluidic chip 30 was expanded to house three aliquoting interfaces formed by the corrugated features 28 (e.g., three such corrugated features 28 formed in a wall) and three assay chambers 114 (FIG. 11A). In this customized microfluidic chip 30, the assay chambers 114 were pre-filled with the RT-LAMP assay solutions containing the primer sets specific to the assigned targets. The assay reagents may also be provided in dry form in some embodiments. Through software level programming, the instruction set was updated to accommodate for the ferrobotic production of three aliquots from a single sample and delivery of each of the aliquots to a designated assay chamber 114 (FIGS. 11B, 11C). The suitability of the platform was validated for multiplexed testing, by successfully differentiating different combinations of spiked input samples (FIG. 11D).

### Competitive Advantages of Ferrobotics

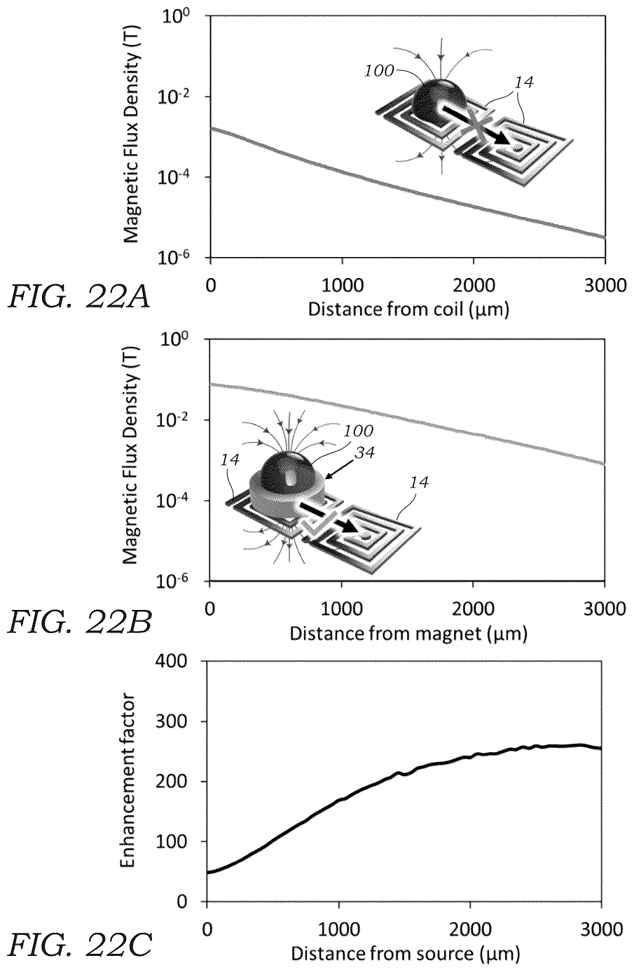

The competitive advantages of the ferrobotic technology are rooted in the electronically programmable nature of the platform, strong, contactless magnetic droplet actuation mechanism that it uniquely employs for liquid handling (which is in principle, battery-operable; FIG. 23B). As such, this technology bypasses the fundamental limitations of magnetic droplet microfluidics approaches that either 1) use complex translational stages (requiring robotic arms for automation) and bulky magnets that are not scalable/portable or 2) use standalone electromagnetic coils to directly actuate the droplets, thus lacking the ample driving forces necessary to execute fluid operations in a rapid and robust manner (leading to two orders of magnitude weaker actuation forces compared to the ferrobotic equivalent; FIGS. 22A-22C).

Furthermore, the employed contactless magnetic actuation mechanism of the ferrobotic technology allows bypassing the reliability issues encountered in EWOD approaches (including surface breakdown, electric charging, and surface hydrophobicity loss caused by ionic droplets). These issues stem from the current EWOD approaches' reliance on high excitation voltages, electric-field-based surface interactions, and specialized hydrophobic surfaces. To circumvent such limitations, entirely different device physics are still being explored for EWOD; albeit their own application for droplet handling and performing complex bioanalytical operations remains nontrivial and yet to be demonstrated. Moreover, unlike the case for the common EWOD devices, the fabrication of ferrobotic microfluidic chips 30 does not involve complex procedures and costly materials. Specifically, the total material cost of a generic ferrobotic microfluidic chip 30 is about two orders of magnitude less than that of an EWOD chip equivalent (Table 2). This cost advantage is especially important for the envisioned application, where the test chips cannot be reused and should be disposed of due to contamination/biohazard. In this setting, the high cost of the hypothetical EWOD chip itself could become a barrier to large-scale frequent testing.

Moreover, it is worth noting that the unique actuation mechanism of the ferrobotic technology renders it advantageous over microfluidic large-scale integrated (mLSI) solutions, especially from the cost, portability, and scalability standpoints. That is because unlike the mLSI solutions that require bulky, expensive, mechanical ancillary equipment to control fluidic operations (e.g., air pumps and pressure regulators to control pneumatic valves), the ferrobotic technology uses electronically-driven PCBs 12 and millimeter-sized magnets 34.

### Considerations for Operational Scalability and Miniaturization

For the demonstrated application herein, scaling up to larger 2D array sizes for manipulating a higher number of samples was limited by sample over-dilution. However, for other applications if such scaling is required, it can be facilitated by either expanding the footprint of the device 10 itself (e.g., to the manufacturing limits of PCB 12) or by employing multiple ferrobotic units and forming a distributed ferrobotic network (leveraging the software-enabled connectivity of the ferrobotic units).

Furthermore, if increase in operational density is desired, trace/component-crowding may naturally pose a challenge. Generally, these challenges can be tackled by 1) utilizing advanced PCB manufacturing processes to minimize the trace width/spacing and increase the number of the board layers for additional degrees of freedom in signal routing; 2) employing proper PCB design/layout techniques; and 3) utilizing advanced microelectronics packaging solutions to minimize the electronic components' footprints. Furthermore, for high-density operations, scaling the number of ferrobots 34 can cause traffic-like issues. For simple planning scenarios, individual ferrobots 34 can be assigned to perform multiple proximal tasks to minimize ferrobot crowding (by exploiting ferrobot's ability to rapidly perform the desired operations). For advanced planning scenarios, specialized navigation planning models and algorithms should be developed to optimize the ferrobotic productivity, while accounting for constraints such as “safety distance” (defined as the minimum distance that should be maintained to avoid inter-ferrobotic magnetic interference, which in this case is ~10 mm). To this end, readily developed models and algorithms in the field of the Automated Guided Vehicles (AGVs) and swarm robotics—that aim to address challenges of similar nature can be adapted and applied within the framework of the ferrobotic platform.

Further minimization of the droplet volume was limited by the number of copies of the virus in the end test sample Further minimization may require further reduction in microfluidic channel dimensions. Advanced laser patterning, injection molding, hot embossing or soft lithography techniques may be used to render microfluidic channel features with finer resolutions.

TABLE 5
Table 5: Kinematics of ferrofluid droplets in different oil environments. Non-
dimensional numbers are assuming a characteristic length scale of ~1 mm.

| | Mineral | | FC-40 | Novec | Novec 7500 |
|---|---|---|---|---|---|
| | Oil | FC-40 | (w/surfactant) | 7500 | (w/surfactant) |
| Maximum velocity (cm/s) | 0.4 | 2.1 | 2.9 | 4.1 | 5.5 |
| Surface tension (mN/m) | 30 | 16 | 12 | 16.2 | 7 |
| Viscosity (mPa · s) | 24.1 | 3.6 | 4.0 | 1.25 | 1.18 |
| Oil density (g/mL) | 0.85 | 1.85 | 1.85 | 1.61 | 1.61 |
| Capillary number | 0.0032 | 0.0047 | 0.0097 | 0.0032 | 0.0092 |
| Reynolds number | 0.14 | 10.8 | 13.4 | 52.8 | 75.0 |

While embodiments of the present invention have been shown and described, various modifications may be made without departing from the scope of the present invention. For example, while the LAMP-based assays were utilized herein, the platform may be used with other nucleic assay amplification formats (e.g., RT-PCR, etc.). In addition, the platform may be used with other assays beyond NAATs. This includes, for example, cell-based assays, enzymatic assays, and electrochemical assays by way of illustration and not limitation. The invention, therefore, should not be limited, except to the following claims, and their equivalents.

1. A ferrofluidic fluid assay device comprising:
a first substrate having a plurality of individually addressable coils formed therein or thereon;
a second substrate disposed adjacent to the first substrate and separated by a gap, the second substrate containing therein one or more volumes of ferrofluid, one or more sample holding chambers or regions, one or more corrugated features for defining sub-volume(s) of the ferrofluid and/or sample, and one or more assay chambers holding an assay solution or assay reagent therein;
one or more permanent magnets interposed in the gap formed between the first substrate and the second substrate, wherein the one or more permanent magnets are moveable within the gap; and
a power source and control circuitry electrically connected to the individually addressable coils and configured to selectively drive current through one or more of the individually addressable coils to move the one or more permanent magnets and the one or more volumes of ferrofluid.

2. The device of claim 1, further comprising a computing device or microcontroller that selectively drives current through one or more of the individually addressable coils in accordance with a script or program.

3. The device of claim 1, further comprising one or more optical sensing modules comprising a light source and an optical sensor configured to read an optical output from the one or more assay chambers.

4. The device of claim 1, further comprising one or more heaters disposed on the first substrate or the second substrate.

5. The device of claim 1, wherein the second substrate comprises a plurality of corrugated features and a plurality of assay chambers.

6. The device of claim 5, wherein the plurality of assay chambers comprises different assay types.

7. The device of claim 1, wherein reverse transcription loop mediated isothermal amplification (RT-LAMP) assay solution or assay reagents are located in the one or more assay chambers.

8. The device of claim 1, wherein the plurality of individually addressable coils comprise a plurality of adjacent coils that are cyclically driven to move a plurality of permanent magnets and associated volumes of ferrofluid in close proximity to effectuate mixing of the volumes of ferrofluid.

9. The device of claim 1, further comprising an array of sample holding chambers or regions disposed on or in the second substrate and arranged in rows and columns.

10. The device of claim 9, wherein the array of sample holding chambers or regions comprises a 3×3 array or a 4×4 array.

11. The device of claim 10, wherein each row and column is associated with a separate assay chamber.

12. The device of claim 9, wherein each sample holding chamber or region is associated with one or more corrugated features for aliquoting the respective samples into smaller sub-volumes of the sample.

13. The device of claim 12, wherein each sample holding chamber or region is associated with a plurality of corrugated features for aliquoting the respective samples into a plurality of smaller sub-volumes of the sample.

14. The device of claim 1, further comprising one or more trench chambers including a trench recess formed therein.

15. The device of claim 1, wherein the ferrofluidic fluid assay device is a handheld portable device and includes a screen or display.

16. A method of using the device of claim 1, comprising:
loading sample(s) into the one or more sample holding chambers or regions; and
driving current through the one or more of the plurality of individually addressable coils to move the one or more permanent magnets to perform one or more unit operations on the one or more volumes of ferrofluid, wherein the unit operations comprise moving the one or more volumes of ferrofluid across a surface of the second substrate, merging the one or more volumes of ferrofluid with the sample(s), forming a plurality of sub-volumes of ferrofluid mixed with the sample(s) with the one or more corrugated features, mixing the one or more ferrofluid volumes and sample(s), pooling the one or more volumes of ferrofluid and sample(s), and moving the one or more pooled volumes of ferrofluid and sample(s) to the one or more assay chambers.

17. The method of claim 16, wherein the sample loading operation comprises pooling a plurality of separate samples and loading the pooled volume in the one or more sample holding chambers or regions.

18. The method of claim 16, wherein a plurality of sub-volumes of ferrofluid mixed with the sample(s) are moved into an array arrayed in rows and columns on the second substrate each holding a different sample.

19. The method of claim 18, wherein a first set of sub-volumes of ferrofluid holding different samples are pooled with other samples along the same row and a second set of sub-volumes holding different samples are pooled with other samples along the same column.

20. The method of claim 19, wherein the pooled sub-volumes of ferrofluid and samples of each row and column are moved to the one or more assay chambers.

21. The method of claim 16, further comprising optically interrogating the one or more assay chambers.

22. The method of claim 21, wherein optically interrogating the one or more assay chambers comprises analyzing the color of the one or more assay chambers.

23. The method of claim 22, wherein optically interrogating the one or more assay chambers comprises illuminating the one or more assay chambers and analyzing the color of the one or more assay chambers with an optical sensing module or optical detector.

24. The method of claim 16, wherein driving current through the plurality of individually addressable coils is performed in accordance with a program or script executed by software contained in a computing device or microcontroller.

25. The method of claim 16, wherein a plurality of assay chambers are loaded with different assay solutions or assay reagents.

26. A method of using the device of claim 14, comprising:
mixing a lysis solution containing magnetic beads with a sample in the one or more trench chambers by movement of the one or more permanent magnets;
removing the lysis solution from the one or more trench chambers by movement of the one or more permanent magnets, wherein the magnetic beads remain in the one or more trench chambers;
mixing a wash solution with the magnetic beads in the one or more trench chambers by movement of the one or more permanent magnets;
removing the wash solution from the one or more trench chambers by movement of the one or more permanent magnets, wherein the magnetic beads remain in the one or more trench chambers;
mixing an elution buffer with the magnetic beads in the one or more trench chambers by movement of the one or more permanent magnets;
removing the elution buffer from the one or more trench chambers by movement of the one or more permanent magnets, wherein the magnetic beads remain in the one or more trench chambers;
transporting the elution buffer to one or more assay chambers by movement of the one or more permanent magnets; and
optically interrogating the one or more assay chambers to view a colorimetric change or a fluorescence signal or output.
